(19) United States
(12) Patent Application Publication
(10) Pub. No.: US 2024/0092831 A1
(43) Pub. Date: Mar. 21, 2024

(54) Proteasome Inhibitors

(71) Applicant: Mayo Foundation for Medical Education and Research, Rochester, MN (US)

(72) Inventors: Asher Alban Akmal Chanan-Khan, Jacksonville, FL (US); Aneel Paulus, Ocala, FL (US); Thomas R. Caulfield, Jacksonville, FL (US)

(21) Appl. No.: 17/768,199

(22) PCT Filed: Oct. 9, 2020
(86) PCT No.: PCT/US2020/055010

§ 371 Date: Apr. 11, 2022

Related U.S. Application Data
(60) Provisional application No. 62/914,221, filed on Oct. 11, 2019.
(60) Provisional application No. 62/914,218, filed on Oct. 11, 2019.

(51) Int. Cl. C07K 5/107; A61P 35/00; C07K 5/062

(52) U.S. Cl. CPC C07K 5/1016; A61P 35/00; C07K 5/06043; A61K 38/00

(57) ABSTRACT

In some embodiments, the present disclosure provides a compound of Formula (I) as described herein, or a pharmaceutically acceptable salt thereof. In some embodiments, the present disclosure provides a compound of Formula (II) as described herein, or a pharmaceutically acceptable salt thereof. Pharmaceutical compositions comprising the compound of Formula (I) or Formula (II), and methods of treating cancer using the compound of Formula (I) or Formula (II) are also provided.

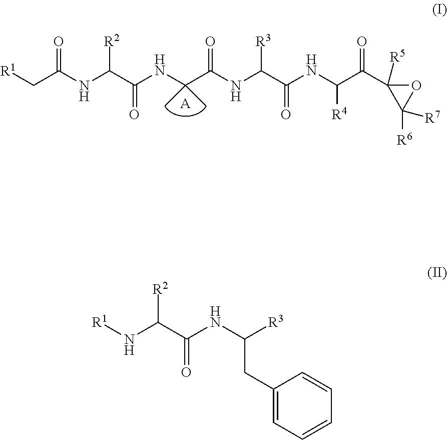

### CLAIM OF PRIORITY

This application claims priority to U.S. Provisional Patent Application Ser. No. 62/914,218, filed on Oct. 11, 2019, and U.S. Provisional Patent Application Ser. No. 62/914,221, filed on Oct. 11, 2019, the entire contents of which are hereby incorporated by reference.

### FEDERALLY SPONSORED RESEARCH AND DEVELOPMENT

This invention was made with government support under grant number CA186781 awarded by National Institutes of Health. The government has certain rights in the invention.

### TECHNICAL FIELD

This invention relates to organic compounds, and more particularly to tetrapeptides and 5-amino-2-benzyl-4-oxopentanal compounds useful in treating cancer such as multiple myeloma.

### BACKGROUND

Cancer is one of the leading causes of death in contemporary society. The numbers of new cancer cases and deaths is increasing each year. Currently, cancer incidence is 454.8 cases of cancer per 100,000 men and women per year, while cancer mortality is 71.2 cancer deaths per 100,000 men and women per year. Pharmacological interventions that are safe over the long term may improve cancer treatment and decrease cancer mortality.

### SUMMARY

The present disclosure describes a series of chemical compounds that inhibit enzymatic activity of the B5 (PSMB5) and B1i (PSMB9) catalytic subunits within the 20S mammalian proteasome. The results presented herein show that both PSMB5 and PSBM9 are cooperatively utilized by malignant. B/plasma cells after chronic exposure to bortezomib or ixazomib (FDA-approved proteasome inhibitors). Both bortezomib and ixazomib are designed to target (PSMB5), and have significantly lower affinity towards PSMB9. In contrast, the compounds described herein are dual PSMB5-PSMB9 inhibitors.

In one general aspect, the present disclosure provides a compound of Formula (I):

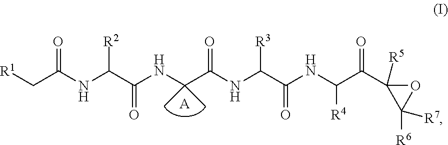

or a pharmaceutically acceptable salt thereof, wherein:

R¹ is 4-10 membered heterocycloalkyl, optionally substituted with 1, 2, or 3 substituents independently selected from R^{a};

R², R³, and R⁴ are each independently selected from H, hydroxymethyl, (imidazol-4-yl)methyl, 3-guanidinopropyl, 4-aminobutyl, 3-aminopropyl, carboxymethyl, 2-carboxyethyl, 1-hydoxyethyl, 2-hydroxyethyl, carbamylmethyl, 2-carbamylethyl, thiomethyl, 2-thioethyl, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;

ring A is selected from 4-7 membered heterocycloalkyl ring and C₃₋₇ cycloalkyl ring, each of which is optionally substituted by 1, 2, or 3 substituents independently selected from R^{a};

R⁵, R⁶, and R⁷ are each independently selected from H and C₁₋₃ alkyl, wherein said C₁₋₃ alkyl is optionally substituted with 1, 2, or 3 substituents independently selected from R^{a}; and

each R^{a} is independently selected from C₁₋₃ alkyl, C₁₋₃ haloalkyl, OH, NO₂, CN, halo, C₁₋₃ alkoxy, C₁₋₃ haloalkoxy, NH₂, C₁₋₃ alkylamino and di(C₁₋₃ alkyl)amino.

In some embodiments, R¹ is selected from aziridinyl, pyrrolidinyl, tetrahydrofuranyl, tetrahydrothiofuranyl, morpholinyl, thiomorpholinyl, piperidinyl, piperazinyl, and tetrahydropyranyl, each of which is optionally substituted with 1, 2, or 3 substituents independently selected from R^{a}.

In some embodiments, R¹ is selected from morpholinyl, thiomorpholinyl, piperidinyl, piperazinyl, and tetrahydropyranyl, each of which is optionally substituted with 1, 2, or 3 substituents independently selected from R^{a}.

In some embodiments, R¹ is morpholinyl, optionally substituted with 1, 2, or 3 substituents independently selected from R^{a}.

In some embodiments, the compound of Formula (I) has formula:

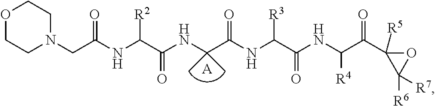

or a pharmaceutically acceptable salt thereof.

In some embodiments, R², R³, and R⁴ are each independently selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl.

In some embodiments:

R⁴ is isobutyl; and

R² and R³ are each independently selected from H, phenylmethyl, 2-phenylethyl.

In some embodiments:

R² is 2-phenylethyl; and

R³ is phenylmethyl.

In some embodiments:

R² is 2-phenylethyl; and

R³ is H.

In some embodiments, R⁵, R⁶, and R⁷ are each independently selected from H and C₁₋₃ alkyl.

In some embodiments:

R⁵ is C₁₋₃ alkyl; and

R⁶ and R⁷ are each H.

In some embodiments, the compound of Formula (I) has formula:

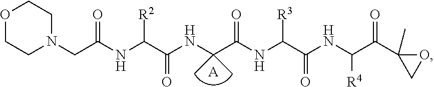

or a pharmaceutically acceptable salt thereof.

In some embodiments, ring A is 4-7 membered heterocycloalkyl ring, optionally substituted by 1, 2, or 3 substituents independently selected from R^{a}.

In some embodiments, ring A is selected from aziridinyl, oxetanyl, pyrrolidinyl, tetrahydrofuranyl, tetrahydrothiofuranyl, morpholinyl, thiomorpholinyl, piperidinyl, piperazinyl, tetrahydropyranyl, 2-azaspiro[3.3]heptyl, and 2-oxaspiro[3.3]heptyl, each of which is optionally substituted by 1, 2, or 3 substituents independently selected from R^{a}.

In some embodiments, ring A is C₃₋₇ cycloalkyl ring, optionally substituted by 1, 2, or 3 substituents independently selected from R^{a}.

In some embodiments, ring A is selected from cyclopropyl, cyclobutyl, cyclopentyl, cyclohexyl, cycloheptyl, and spiro[3.3]heptyl, each of which is optionally substituted by 1, 2, or 3 substituents independently selected from R^{a}.

In some embodiments, ring A is selected from cyclohexyl and spiro[3.3]heptyl, each of which is optionally substituted by 1, 2, or 3 substituents independently selected from R^{a}.

In some embodiments, each R^{a} is independently selected from halo, C₁₋₃ alkyl, C₁₋₃ haloalkyl, C₁₋₃ alkoxy, and C₁₋₃ haloalkoxy.

In some embodiments, each R^{a} is halo.

In some embodiments, the compound of Formula (I) has formula:

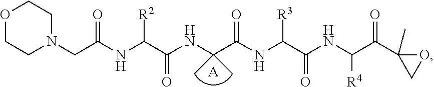

or a pharmaceutically acceptable salt thereof, wherein:

R², R³, and R⁴ are each independently selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;

ring A is C₃₋₇ cycloalkyl ring, optionally substituted by 1, 2, or 3 substituents independently selected from R^{a}; and

each R^{a} is independently selected from halo, C₁₋₃ alkyl, C₁₋₃ haloalkyl, C₁₋₃ alkoxy, and C₁₋₃ haloalkoxy.

In some embodiments:

R⁴ is isobutyl;

R² and R³ are each independently selected from H, phenylmethyl, and 2-phenylethyl;

and ring A is selected from cyclohexyl and spiro[3.3]heptyl, each of which is optionally substituted by 1, 2, or 3 halo.

In some embodiments, the compound of Formula (I) is selected from any one of the following compounds:

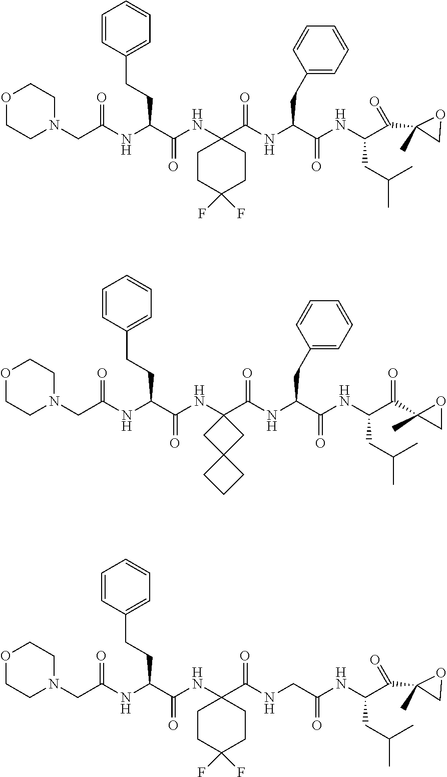 [SMILES: CC(C)C[C@H](NC(=O)[C@H](Cc1ccccc1)NC(=O)C1(NC(=O)[C@H](CCc2ccccc2)NC(=O)CN2CCOCC2)CCC(F)(F)CC1)C(=O)[C@@]1(C)CO1 ; CC(C)C[C@H](NC(=O)[C@H](Cc1ccccc1)NC(=O)C1(NC(=O)[C@H](CCc2ccccc2)NC(=O)CN2CCOCC2)CC2(CCC2)C1)C(=O)[C@@]1(C)CO1 ; CC(C)C[C@H](NC(=O)CNC(=O)C1(NC(=O)[C@H](CCc2ccccc2)NC(=O)CN2CCOCC2)CCC(F)(F)CC1)C(=O)[C@@]1(C)CO1]

or a pharmaceutically acceptable salt thereof.

In another general aspect, the present disclosure provides a pharmaceutical composition comprising a compound of Formula (I) as described herein, or a pharmaceutically acceptable salt thereof, and a pharmaceutically acceptable carrier.

In another general aspect, the present disclosure provides a method of inhibiting enzymatic activity of a subunit β5 and a subunit β1i of a proteasome in a cell, the method comprising contacting the cell with an effective amount of a compound of Formula (I) as described herein, or a pharmaceutically acceptable salt thereof. In some embodiments, the method comprises contacting the cell in vitro, in vivo, or ex vivo.

In another general aspect, the present disclosure provides a method of treating cancer in a subject, the method comprising administering to the subject a therapeutically effective amount of a compound of Formula (I) as described herein, or a pharmaceutically acceptable salt thereof, or a pharmaceutical composition comprising same.

In some embodiments, the cancer is multiple myeloma.

In some embodiments, the method further comprises administering to the subject at least one additional anti-cancer agent, or a pharmaceutically acceptable salt thereof.

In some embodiments, the additional anti-cancer agent is a proteasome inhibitor.

In some embodiments, the proteasome inhibitor is selected from bortezomib, carfilzomib, and ixazomib, or a pharmaceutically acceptable salt thereof.

In some embodiments, the present disclosure provides a compound of Formula (II):

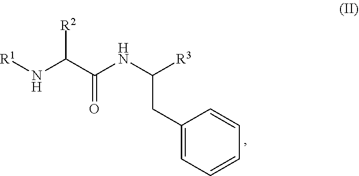

or a pharmaceutically acceptable salt thereof, wherein:

R¹ is selected from C(O)R^{a1}, C(O)OR^{a1}, and S(O)₂R^{a1};

R² is selected from H, hydroxymethyl, (imidazol-4-yl)methyl, 3-guanidinopropyl, 4-aminobutyl, 3-aminopropyl, carboxymethyl, 2-carboxyethyl, 1-hydoxyethyl, 2-hydroxyethyl, carbamylmethyl, 2-carbamylethyl, thiomethyl, 2-thioethyl, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;

R³ is selected from C(O)R^{b1}, CN, C(═NR^{e1})R^{b1}, and a group of formula:

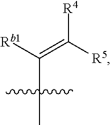

R⁴ and R⁵ are each independently selected from C(O)OR^{c1}, S(O)₂R^{c1}, C(O)R^{c1}, C(O)NR^{c1}R^{d1}, S(O)₂NR^{c1}R^{d1}, CN and NO₂;

each R^{a1} is selected from C₁₋₆ alkyl, C₁₋₆ haloalkyl, C₂₋₆ alkenyl, C₂₋₆ alkynyl, C₆₋₁₀ aryl, 5-14 membered heteroaryl, C₆₋₁₀ aryl-C₁₋₄ alkylene, and (5-10 membered heteroaryl)-C₁₋₄ alkylene, each of which is optionally substituted with 1, 2, or 3 substituents independently selected from R^{g};

each R^{b1} is selected from H, C₁₋₃ alkyl, and C₁₋₃ haloalkyl;

R^{e1} is selected from H, C₁₋₃ alkyl, C₁₋₃ alkoxy, OH, and CN;

each R^{c1} and R^{d1} are independently selected from H, C₁₋₃ alkyl, and C₁₋₃ haloalkyl; and

each R^{g} is independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆alkyl)amino.

In some embodiments, each R^{a1} is selected from C₁₋₆ alkyl, C₁₋₆ haloalkyl, C₆₋₁₀ aryl, C₆₋₁₀ aryl-C₁₋₄ alkylene, 5-14 membered heteroaryl, and (5-10 membered heteroaryl)-C₁₋₄ alkylene, each of which is optionally substituted with 1 or 2 substituents independently selected from R^{g}.

In some embodiments, each R^{a1} is selected from C₁₋₆ alkyl, C₆₋₁₀ aryl, C₆₋₁₀ aryl-C₁₋₄ alkylene, and 5-14 membered heteroaryl, each of which is optionally substituted with 1 or 2 substituents independently selected from R^{g}.

In some embodiments, R¹ is C(O)R^{a1}.

In some embodiments, R¹ is C(O)OR^{a1}.

In some embodiments, R^{a1} is C₆₋₁₀ aryl-C₁₋₄ alkylene, optionally substituted with 1 or 2 substituents independently selected from R^{g}.

In some embodiments, R¹ is S(O)₂R^{a1}.

In some embodiments, R^{a1} is C₁₋₆ alkyl, optionally substituted with R^{g}.

In some embodiments, R^{g} is selected from halo, C₁₋₆ alkyl, C₁₋₄ haloalkyl, C₁₋₆ alkoxy, and C₁₋₆ haloalkoxy.

In some embodiments, R² is selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl.

In some embodiments, R² is selected from isopropyl, isobutyl, and phenylmethyl.

In some embodiments, R² is isopropyl.

In some embodiments, R³ is C(O)R^{b1}.

In some embodiments, R³ is C(═NR^{e1})R^{b1}.

In some embodiments, R³ is a group of formula:

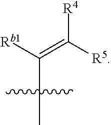

In some embodiments, R^{b1} is selected from C₁₋₃ alkyl and C₁₋₃ haloalkyl.

In some embodiments, the compound of Formula (II) has formula:

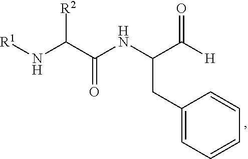

or a pharmaceutically acceptable salt thereof.

In some embodiments, the compound of Formula (II) has formula:

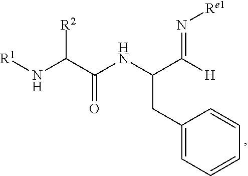

or a pharmaceutically acceptable salt thereof.

In some embodiments, R^{e1} is selected from OH and C₁₋₃ alkoxy.

In some embodiments, the compound of Formula (II) has formula:

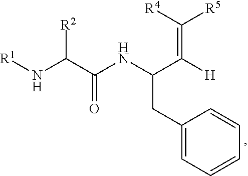

or a pharmaceutically acceptable salt thereof.

In some embodiments, R⁴ and R⁵ are each independently selected from C(O)OR^{c1}, CN, and NO₂.

In some embodiments, R^{c1} is selected from C₁₋₃ alkyl and C₁₋₃ haloalkyl.

In some embodiments, the compound of Formula (II) is selected from any one of the following compounds:

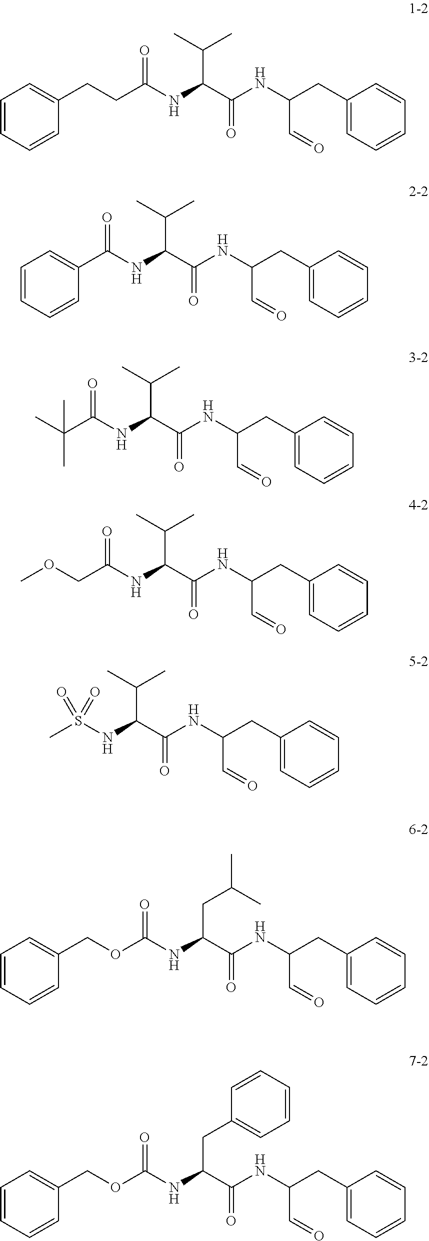

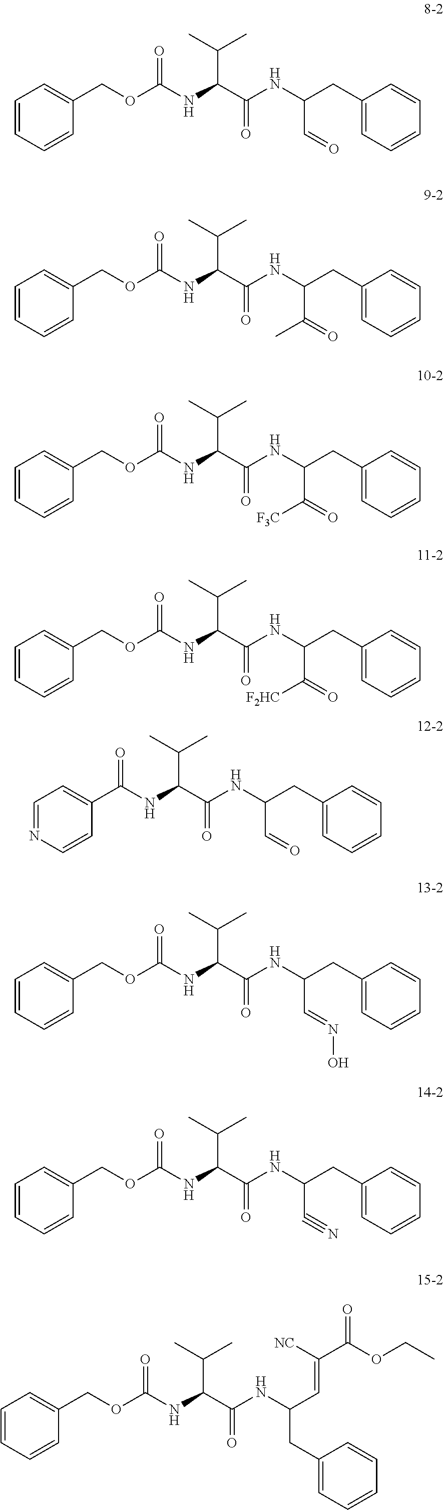

or a pharmaceutically acceptable salt thereof.

In some embodiments, the present disclosure provides a pharmaceutical composition comprising a compound of Formula (II) as described herein, or a pharmaceutically acceptable salt thereof, and a pharmaceutically acceptable carrier.

In some embodiments, the present disclosure provides a method of inhibiting enzymatic activity of a subunit β5 and a subunit β1i of a proteasome in a cell, the method comprising contacting the cell with an effective amount of a compound of Formula (II) as described herein, or a pharmaceutically acceptable salt thereof. In some embodiments, the method comprises contacting the cell in vitro, in vivo, or ex vivo.

In some embodiments, the present disclosure provides a method of treating cancer in a subject, the method comprising administering to the subject a therapeutically effective amount of a compound of Formula (II) as described herein, or a pharmaceutically acceptable salt thereof, or a pharmaceutical composition as described herein.

In some embodiments, the cancer is multiple myeloma.

In some embodiments, the method further comprises administering to the subject at least one additional anti-cancer agent, or a pharmaceutically acceptable salt thereof.

In some embodiments, the additional anti-cancer agent is a proteasome inhibitor.

In some embodiments, the proteasome inhibitor is selected from bortezomib, carfilzomib, and ixazomib, or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A contains line plots showing that combined RNA-i of PSMB9 and PSMB5 reduces multiple myeloma cell proliferation (OPM2, bortezomib-sensitive cells).

FIG. 1B contains line plots showing that combined RNA-i of PSMB9 and PSMB5 reduces multiple myeloma cell proliferation (OPM2/BR, bortezomib-resistant cells).

FIG. 2A contains images showing Compound 1 bound as a ligand to PSMB5 and PSMB9 and corresponding binding energies.

FIG. 2B contains a line plot showing Compound 1 inhibitory activity in bortezomib-sensitive cell lines.

FIG. 2C contains a line plot showing Compound 1 inhibitory activity in bortezomib-resistant cell lines.

FIG. 2D contains bar graphs showing PSMB5 and PSMB9 enzyme inhibition by Compound 1 in bortezomib-sensitive cells.

FIG. 2E contains bar graphs showing PSMB5 and PSMB9 enzyme inhibition by Compound 1 in bortezomib-resistant cells.

FIG. 2F contains images showing that Compound 1 induces apoptosis and ER stress in WM cells developed from a bortezomib-resistant WM patient.

FIG. 2G contains a Western blot image showing that apoptotic cell death caused by Compound 1 was accompanied by induction of the ER-stress response demonstrated by a dose-dependent increase in XBP-1 and XBP-1s proteins and polyubiquitinated protein.

FIG. 3 contains Western blot images showing that PSMB9 protein expression is upregulated in bortezomib-resistant multiple myeloma cells.

FIG. 4A contains a Western Blot image showing that PSMB9 is markedly expressed in primary tumor cells from bortezomib-resistant myeloma patients.

FIG. 4B contains a histogram showing that PSMB9 is markedly expressed in primary tumor cells from bortezomib-resistant myeloma patients.

FIG. 5 contains a line plot showing that PSMB9 mRNA is significantly lower in patients who respond to bortezomib-based therapy.

FIG. 6 contains a line plot showing that PSMB9 mRNA does not correlate with response in patients on lenalidomide-based treatment.

FIG. 7 contains data showing that genetic interference of PSBM9 is lethal to multiple myeloma cells.

FIG. 8 contains images showing binding affinity of dual-β5/β1i small molecule inhibitors (or current PI) to PSMB5 and PSMB9.

FIG. 9 contains images containing Compound 1 bound to PSBM5 and PSBM9.

FIG. 10 contains bar graphs showing in vitro enzymatic and anti-tumor potency of dual-β5/β1i small molecule inhibitors of the present disclosure.

### DETAILED DESCRIPTION

### Therapeutic Compounds

The present application provides, inter alia, a compound of Formula (I):

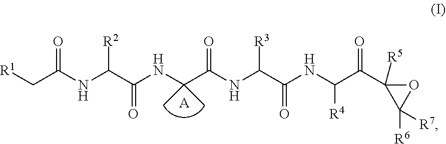

or a pharmaceutically acceptable salt thereof, wherein R¹, R², R³, R⁴, R⁵, R⁶, R⁷, and ring A are as described herein.

In some embodiments:

R¹ is 4-10 membered heterocycloalkyl, optionally substituted with 1, 2, or 3 substituents independently selected from R^{a};

R², R³, and R⁴ are each independently selected from H, hydroxymethyl, yl)methyl, 3-guanidinopropyl, 4-aminobutyl, 3-aminopropyl, carboxymethyl, 2-carboxyethyl, 1-hydoxyethyl, 2-hydroxyethyl, carbamylmethyl, 2-carbamylethyl, thiomethyl, 2-thioethyl, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-15 yl)methyl;

ring A is selected from 4-7 membered heterocycloalkyl ring and C₃₋₇ cycloalkyl ring, each of which is optionally substituted by 1, 2, or 3 substituents independently selected from R^{a};

R⁵, R⁶, and R⁷ are each independently selected from H and C₁₋₃ alkyl, wherein said C₁₋₃ alkyl is optionally substituted with 1, 2, or 3 substituents independently selected from R^{a}; and

each R^{a} is independently selected from C₁₋₃ alkyl, C₁₋₃ haloalkyl, OH, NO₂, CN, halo, C₁₋₃ alkoxy, C₁₋₃ haloalkoxy, NH₂, C₁₋₃ alkylamino and di(C₁₋₃ alkyl)amino.

In some embodiments, R¹ is selected from aziridinyl, pyrrolidinyl, tetrahydrofuranyl, tetrahydrothiofuranyl, morpholinyl, thiomorpholinyl, piperidinyl, piperazinyl, and tetrahydropyranyl, each of which is optionally substituted with 1, 2, or 3 substituents independently selected from R^{a}.

In some embodiments, R¹ is selected from morpholinyl, thiomorpholinyl, piperidinyl, piperazinyl, and tetrahydropyranyl, each of which is optionally substituted with 1, 2, or 3 substituents independently selected from R^{a}.

In some embodiments, R¹ is aziridinyl, optionally substituted with 1, 2, or 3 substituents independently selected from R^{a}.

In some embodiments, R¹ is pyrrolidinyl, optionally substituted with 1, 2, or 3 substituents independently selected from R^{a}.

In some embodiments, R¹ is tetrahydrofuranyl, optionally substituted with 1, 2, or 3 substituents independently selected from R^{a}.

In some embodiments, R¹ is thiomorpholinyl, optionally substituted with 1, 2, or 3 substituents independently selected from R^{a}.

In some embodiments, R¹ is piperidinyl, optionally substituted with 1, 2, or 3 substituents independently selected from R^{a}.

In some embodiments, R¹ is piperazinyl, optionally substituted with 1, 2, or 3 substituents independently selected from R^{a}.

In some embodiments, R¹ is tetrahydropyranyl, optionally substituted with 1, 2, or 3 substituents independently selected from R^{a}.

In some embodiments, R¹ is morpholinyl, optionally substituted with 1, 2, or 3 substituents independently selected from R^{a}.

In some embodiments, R¹ is selected from any one of the following groups:

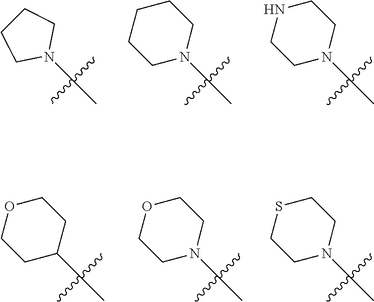 [SMILES: *N1CCCC1 ; *N1CCCCC1 ; *N1CCNCC1 ; *C1CCOCC1 ; *N1CCOCC1 ; *N1CCSCC1]

each of which is optionally substituted with 1, 2, or 3 substituents independently selected from R^{a}.

In some embodiments, R¹ is a group of formula:

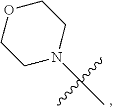 [SMILES: *N1CCOCC1]

which is optionally substituted with 1, 2, or 3 substituents independently selected from R^{a}.

In some embodiments, R², R³, and R⁴ are each independently selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl.

In some embodiments, R², R³, and R⁴ are each independently selected from H, methyl, ethyl, n-propyl, isopropyl, sec-butyl, isobutyl, n-butyl, n-hexyl, phenylmethyl, and 2-phenylethyl.

In some embodiments, R², R³, and R⁴ are each independently selected from H, isobutyl, phenylmethyl, and 2-phenylethyl.

In some embodiments, R² is selected from H, methyl, ethyl, n-propyl, isopropyl, sec-butyl, isobutyl, n-butyl, n-hexyl, phenylmethyl, and 2-phenylethyl. In some embodiments, R² is H. In some embodiments, R² is selected from methyl, ethyl, n-propyl, isopropyl, sec-butyl, isobutyl, n-butyl, n-hexyl, phenylmethyl, and 2-phenylethyl. In some embodiments, R² is selected from isobutyl, phenylmethyl, and 2-phenylethyl. In some embodiments, R² is isobutyl. In some embodiments, R² is phenylmethyl. In some embodiments, R² is 2-phenylethyl.

In some embodiments, R³ is selected from H, methyl, ethyl, n-propyl, isopropyl, sec-butyl, isobutyl, n-butyl, n-hexyl, phenylmethyl, and 2-phenylethyl. In some embodiments, R³ is H. In some embodiments, R³ is selected from methyl, ethyl, n-propyl, isopropyl, sec-butyl, isobutyl, n-butyl, n-hexyl, phenylmethyl, and 2-phenylethyl. In some embodiments, R³ is selected from isobutyl, phenylmethyl, and 2-phenylethyl. In some embodiments, R³ is isobutyl. In some embodiments, R³ is phenylmethyl. In some embodiments, R³ is 2-phenylethyl.

In some embodiments, R⁴ is selected from H, methyl, ethyl, n-propyl, isopropyl, sec-butyl, isobutyl, n-butyl, n-hexyl, phenylmethyl, and 2-phenylethyl. In some embodiments, R⁴ is H. In some embodiments, R⁴ is selected from methyl, ethyl, n-propyl, isopropyl, sec-butyl, isobutyl, n-butyl, n-hexyl, phenylmethyl, and 2-phenylethyl. In some embodiments, R⁴ is selected from isobutyl, phenylmethyl, and 2-phenylethyl. In some embodiments, R⁴ is isobutyl. In some embodiments, R⁴ is phenylmethyl. In some embodiments, R⁴ is 2-phenylethyl.

In some embodiments, R⁴ is isobutyl; and R 2 and R³ are each independently selected from H, phenylmethyl, 2-phenylethyl. In some embodiments, R⁴ is isobutyl, R² is 2-phenylethyl; and R³ is phenylmethyl. In some embodiments, R⁴ is isobutyl; R³ is 2-phenylethyl; and R² is phenylmethyl. In some embodiments, R⁴ is isobutyl; R² is 2-phenylethyl; and R³ is H. In some embodiments, R⁴ is isobutyl; R³ is 2-phenylethyl; and R² is H. In some embodiments, R⁴ is isobutyl; R² is phenylmethyl; and R³ is H. In some embodiments, R⁴ is isobutyl; R³ is phenylmethyl; and R² is H.

In some embodiments, R⁵, R⁶, and R⁷ are each independently selected from H and C₁₋₃ alkyl. In some embodiments, R⁵ is selected from H and C₁₋₃ alkyl. In some embodiments, R⁵ is H. In some embodiments, R⁵ is C₁₋₃ alkyl. In some embodiments, R⁶ is selected from H and C₁₋₃ alkyl. In some embodiments, R⁶ is H. In some embodiments, R⁶ is C₁₋₃ alkyl. In some embodiments, R⁷ is selected from H and C₁₋₃ alkyl. In some embodiments, R⁷ is H. In some embodiments, R⁷ is C₁₋₃ alkyl. In some embodiments, R⁵ is C₁₋₃ alkyl; and R⁶ and R⁷ are each H. In some embodiments, R⁵ is methyl; and R⁶ and R⁷ are each H.

In some embodiments, ring A is 4-7 membered heterocycloalkyl ring, optionally substituted by 1, 2, or 3 substituents independently selected from R^{a}.

In some embodiments, ring A is selected from aziridinyl, oxetanyl, pyrrolidinyl, tetrahydrofuranyl, morpholinyl, thiomorpholinyl, piperidinyl, piperazinyl, tetrahydropyranyl, 2-azaspiro[3.3]heptyl, and 2-oxaspiro[3.3]heptyl, each of which is optionally substituted by 1, 2, or 3 substituents independently selected from R^{a}.

In some embodiments, ring A is selected from any one of the following formulae:

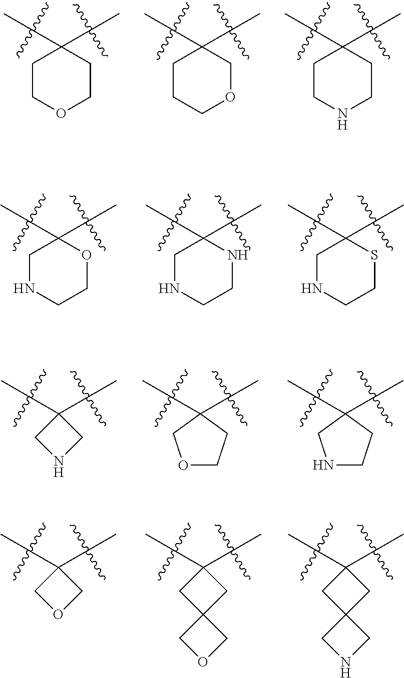

each of which is optionally substituted with 1, 2, or 3 substituents independently selected from R^{a}.

In some embodiments, ring A is C₃₋₇ cycloalkyl ring, optionally substituted by 1, 2, or 3 substituents independently selected from R^{a}.

In some embodiments, ring A is selected from cyclopropyl, cyclobutyl, cyclopentyl, cyclohexyl, cycloheptyl, and spiro[3.3]heptyl, each of which is optionally substituted by 1, 2, or 3 substituents independently selected from R^{a}.

In some embodiments, ring A is selected from cyclohexyl and spiro[3.3]heptyl, each of which is optionally substituted by 1, 2, or 3 substituents independently selected from R^{a}. In some embodiments, ring A is cyclohexyl, optionally substituted by 1, 2, or 3 substituents independently selected from R^{a}. In some embodiments, ring A is spiro[3.3]heptyl, optionally substituted by 1, 2, or 3 substituents independently selected from R^{a}.

In some embodiments, ring A has formula:

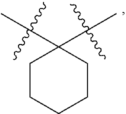 [SMILES: *C1(*)CCCCC1]

which is optionally substituted with 1, 2, or 3 substituents independently selected from R^{a}.

In some embodiments, ring A has formula:

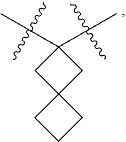 [SMILES: *C1(*)CC2(CCC2)C1]

which is optionally substituted with 1, 2, or 3 substituents independently selected from R^{a}.

In some embodiments, each R^{a} is independently selected from halo, C₁₋₃ alkyl, C₁₋₃ haloalkyl, C₁₋₃ alkoxy, and C₁₋₃ haloalkoxy. In some embodiments, each R^{a} is independently selected from halo, C₁₋₃ alkyl, and C₁₋₃ haloalkyl. In some embodiments, each R^{a} is independently selected from halo and C₁₋₃ haloalkyl. In some embodiments, each R^{a} is halo.

In some embodiments, the compound of Formula (I) has formula:

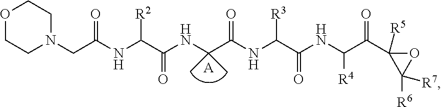

or a pharmaceutically acceptable salt thereof, wherein R², R³, R⁴, R⁵, R⁶, R⁷, and ring A are as described herein.

In some embodiments, the compound of Formula (I) has formula:

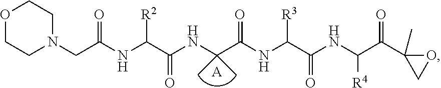

wherein R², R³, R⁴, and ring A are as described herein.

In some embodiments:

R², R³, and R⁴ are each independently selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;

ring A is C₃₋₇ cycloalkyl ring, optionally substituted by 1, 2, or 3 substituents independently selected from R^{a}; and

each R^{a} is independently selected from halo, C₁₋₃ alkyl, C₁₋₃ haloalkyl, C₁₋₃ alkoxy, and C₁₋₃ haloalkoxy.

In some embodiments:

R⁴ is isobutyl;

R² and R³ are each independently selected from H, phenylmethyl, and 2-phenylethyl; and

ring A is selected from cyclohexyl and spiro[3.3]heptyl, each of which is optionally substituted by 1, 2, or 3 halo.

In some embodiments, the compound of Formula (I) is selected from any one fo the following compounds:

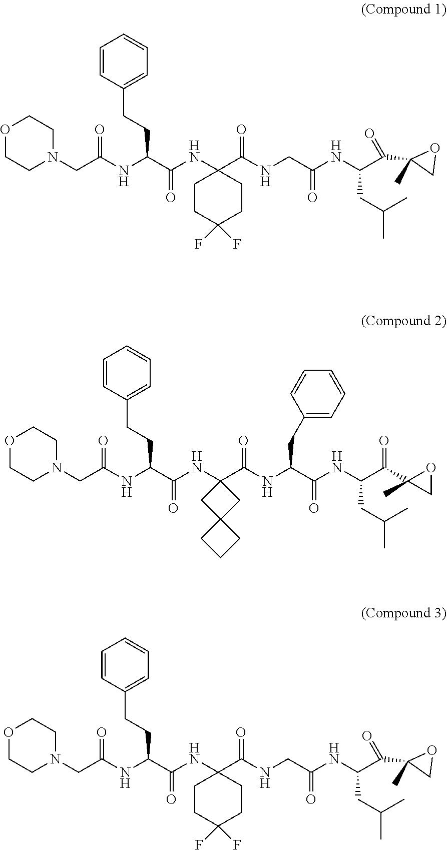

or a pharmaceutically acceptable salt thereof.

In some embodiments, the compound of Formula (I) is not:

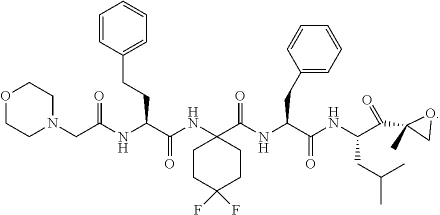 [SMILES: CC(C)C[C@H](NC(=O)[C@H](Cc1ccccc1)NC(=O)C1(NC(=O)[C@H](CCc2ccccc2)NC(=O)CN2CCOCC2)CCC(F)(F)CC1)C(=O)[C@@]1(C)CO1]

The present application also provides, inter alia, a compound of Formula (II):

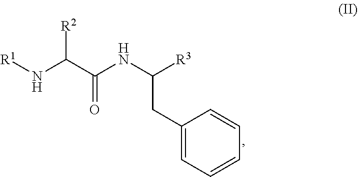

or a pharmaceutically acceptable salt thereof, wherein R¹, R², and R³ are as described herein.

In some embodiments:

R¹ is selected from C(O)R^{a1}, C(O)OR^{a1}, and S(O)₂R^{a1};

R² is selected from H, hydroxymethyl, (imidazol-4-yl)methyl, 3-guanidinopropyl, 4-aminobutyl, 3-aminopropyl, carboxymethyl, 2-carboxyethyl, 1-hydoxyethyl, 2-hydroxyethyl, carbamylmethyl, 2-carbamylethyl, thiomethyl, 2-thioethyl, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;

R³ is selected from C(O)R^{b1}, CN, C(═NR^{e1})R^{b1}, and a group of formula:

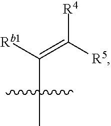

R⁴ and R⁵ are each independently selected from C(O)OR^{c1}, S(O)₂R^{c1}, C(O)R^{c1}, C(O)NR^{c1}R^{d1}, S(O)₂NR^{c1}R^{d1}, CN, and NO₂;

each R^{a1} is selected from C₁₋₆ alkyl, C₁₋₆ haloalkyl, C₂₋₆ alkenyl, C₂₋₆ alkynyl, C₆₋₁₀ aryl, 5-14 membered heteroaryl, C₆₋₁₀ aryl-C₁₋₄ alkylene, and (5-10 membered heteroaryl)-C₁₋₄ alkylene, each of which is optionally substituted with 1, 2, or 3 substituents independently selected from R^{g};

each R^{b1} is selected from H, C₁₋₃ alkyl, and C₁₋₃ haloalkyl;

R^{e1} is selected from H, C₁₋₃ alkyl, C₁₋₃ alkoxy, OH, and CN;

each R^{e1} and R^{d1} are independently selected from H, C₁₋₃ alkyl, and C₁₋₃ haloalkyl; and

each R^{g} is independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino.

In some embodiments, each R^{a1} is selected from C₁₋₆ alkyl, C₁₋₆ haloalkyl, C₆₋₁₀ aryl, C₆₋₁₀ aryl-C₁₋₄ alkylene, 5-14 membered heteroaryl, and (5-10 membered heteroaryl)-C₁₋₄ alkylene, each of which is optionally substituted with 1 or 2 substituents independently selected from R^{g}.

In some embodiments, each R^{a1} is selected from C₁₋₆ alkyl, C₆₋₁₀ aryl, C₆₋₁₀ aryl-C₁₋₄ alkylene, and 5-14 membered heteroaryl, each of which is optionally substituted with 1 or 2 substituents independently selected from R^{g}.

In some embodiments, R^{a1} is C₁₋₆ alkyl, optionally substituted with R^{g}. In some embodiments, R^{a1} is C₁₋₆ alkyl, optionally substituted with C₁₋₆ alkoxy. In some embodiments, is C₆₋₁₀ aryl, optionally substituted with 1 or 2 substituents independently selected from R^{g}. In some embodiments, R^{a1} is C₆₋₁₀ aryl-C₁₋₄ alkylene, optionally substituted with 1 or 2 substituents independently selected from R^{g}. In some embodiments, R^{a1} is 5-14 membered heteroaryl, optionally substituted with 1 or 2 substituents independently selected from R^{g}.

In some embodiments, R¹ is C(O)R^{a1}.

In some embodiments, R¹ is C(O)OR^{a1}. In some aspects of these embodiments, R^{a1} is C₆₋₁₀ aryl-C₁₋₄ alkylene, optionally substituted with 1 or 2 substituents independently selected from R^{g}.

In some embodiments, R¹ is S(O)₂R^{a1}. In some aspects of these embodiments, R^{a1} is C₁₋₆ alkyl, optionally substituted with R^{g}.

In some embodiments, R^{g} is selected from halo, C₁₋₆ alkyl, C₁₋₄ haloalkyl, C₁₋₆ alkoxy, and C₁₋₆ haloalkoxy. In some embodiments, R^{g} is halo. In some embodiments, R^{g} is selected from C₁₋₆ alkyl and C₁₋₄ haloalkyl. In some embodiments, R^{g} is selected from C₁₋₆ alkoxy, and C₁₋₆ haloalkoxy. In some embodiments, R^{g} is selected from C₁₋₆ alkyl and C₁₋₆ alkoxy. In some embodiments, R^{g} is C₁₋₆ alkoxy.

In some embodiments, R² is selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl.

In some embodiments, R² is selected from isopropyl, isobutyl, and phenylmethyl. In some embodiments, R² is isopropyl. In some embodiments, R² is isobutyl. In some embodiments, R² is phenylmethyl.

In some embodiments, R³ is C(O)R^{b1}.

In some embodiments, R³ is C(═NR^{e1})R^{b1}.

In some embodiments, R³ is a group of formula:

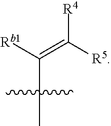

In some embodiments, R^{b1} is selected from C₁₋₃ alkyl and C₁₋₃ haloalkyl. In some embodiments, R^{b1} is C₁₋₃ alkyl. In some embodiments, R^{b1} is C₁₋₃ haloalkyl. In some embodiments, R^{b1} is H.

In some embodiments, R^{e1} is selected from OH and C₁₋₃ alkoxy. In some embodiments, R^{e1} is OH. In some embodiments, R^{e1} is C₁₋₃ alkoxy. In some embodiments, R^{e1} is H. In some embodiments, R^{e1} is CN.

In some embodiments, R⁴ and R⁵ are each independently selected from C(O)OR^{c1}, CN, and NO₂. In some embodiments, R⁴ is C(o)OR^{c1} and R⁵ is CN. In some embodiments, R⁵ is C(O)OR^{c1} and R⁴ is CN.

In some embodiments, R^{e1} and R^{d1} are each independently selected from C₁₋₃ alkyl and C₁₋₃ haloalkyl. In some embodiments, R^{e1} is C₁₋₃ alkyl. In some embodiments, R^{d1} is C₁₋₃ alkyl. In some embodiments, R^{e1} is H and R^{d1} is C₁₋₃ alkyl. In some embodiments, R^{d1} is H and R^{e1} is C₁₋₃ alkyl. In some embodiments, R^{e1} and R^{d1} are each H. In some embodiments, R^{c1} and R^{d1} are each C₁₋₃ alkyl.

In some embodiments, the compound of Formula (II) has formula:

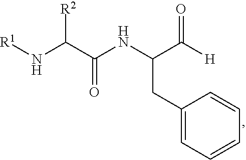

or a pharmaceutically acceptable salt thereof.

In some embodiments, the compound of Formula (II) has formula:

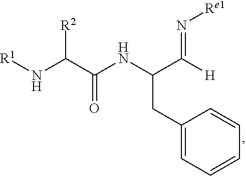

or a pharmaceutically acceptable salt thereof.

In some embodiments, the compound of Formula (II) has formula:

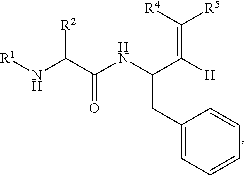

or a pharmaceutically acceptable salt thereof.

In some embodiments:

R¹ is selected from C(O)R^{a1}, C(O)OR^{a1}, and S(O)₂R^{a1};

each R^{a1} is selected from C₁₋₆ alkyl, C₆₋₁₀ aryl, C₆₋₁₀ aryl-C₁₋₄ alkylene, and 5-14 membered heteroaryl, each of which is optionally substituted with 1 or 2 substituents independently selected from R^{g};

R^{g} is selected from halo, C₁₋₆ alkyl, C₁₋₄ haloalkyl, C₁₋₆ alkoxy, and C₁₋₆ haloalkoxy; and

R² is selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl.

In some embodiments:

R¹ is selected from C(O)R^{a1}, C(O)OR^{a1}, and S(O)₂R^{a1};

each R^{a1} is selected from C₁₋₆ alkyl, C₆₋₁₀ aryl, C₆₋₁₀ aryl-C₁₋₄ alkylene, and 5-14 membered heteroaryl, each of which is optionally substituted with 1 or 2 substituents independently selected from R^{g};

R^{g} is selected from halo, C₁₋₆ alkyl, C₁₋₄ haloalkyl, C₁₋₆ alkoxy, and C₁₋₆ haloalkoxy;

R² is selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl; and

R^{e1} is selected from OH and C₁₋₃ alkoxy.

In some embodiments:

R¹ is selected from C(O)R^{a1}, C(O)OR^{a1}, and S(O)₂R^{a1}; each R^{a1} is selected from C₁₋₆ alkyl, C₆₋₁₀ aryl, C₆₋₁₀ aryl-C₁₋₄ alkylene, and 5-14 membered heteroaryl, each of which is optionally substituted with 1 or 2 substituents independently selected from R^{g};

R² is selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;

R⁴ and R⁵ are each independently selected from C(O)OR^{c1}, CN, and NO₂; and

R^{e1} is selected from C₁₋₃ alkyl and C₁₋₃ haloalkyl.

In some embodiments:

R¹ is C(O)R^{a1};

each R^{a1} is selected from C₁₋₆ alkyl, C₆₋₁₀ aryl, C₆₋₁₀ aryl-C₁₋₄ alkylene, and 5-14 membered heteroaryl, each of which is optionally substituted with 1 or 2 substituents independently selected from R^{g};

R^{g} is selected from halo, C₁₋₆ alkyl, C₁₋₄ haloalkyl, C₁₋₆ alkoxy, and C₁₋₆ haloalkoxy; and

R² is selected from isopropyl, isobutyl, and phenylmethyl.

In some embodiments:

R¹ is C(O)OR^{a1};

R^{a1} is C₆₋₁₀ aryl-C₁₋₄ alkylene, optionally substituted with 1 or 2 substituents independently selected from R^{g};

R^{g} is selected from halo, C₁₋₆ alkyl, C₁₋₄ haloalkyl, C₁₋₆ alkoxy, and C₁₋₆ haloalkoxy; and

R² is selected from isopropyl, isobutyl, and phenylmethyl.

In some embodiments:

R¹ is S(O)₂R^{a1};

R^{a1} is C₁₋₆ alkyl, optionally substituted with R^{g};

R^{g} is selected from halo, C₁₋₆ alkyl, C₁₋₄ haloalkyl, C₁₋₆ alkoxy, and C₁₋₆ haloalkoxy; and

R² is selected from isopropyl, isobutyl, and phenylmethyl.

In some embodiments, the compound of Formula (II) is selected from any one of the following compounds:

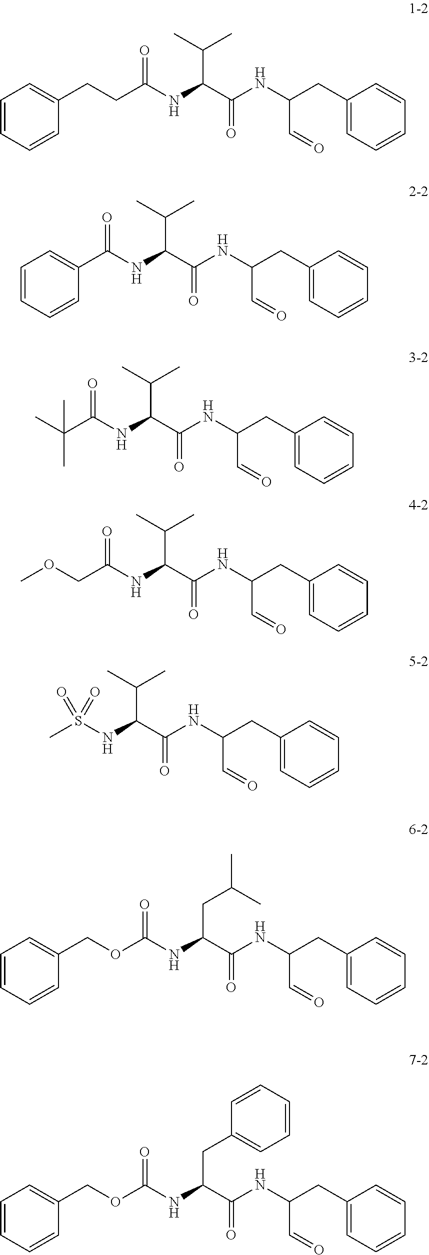

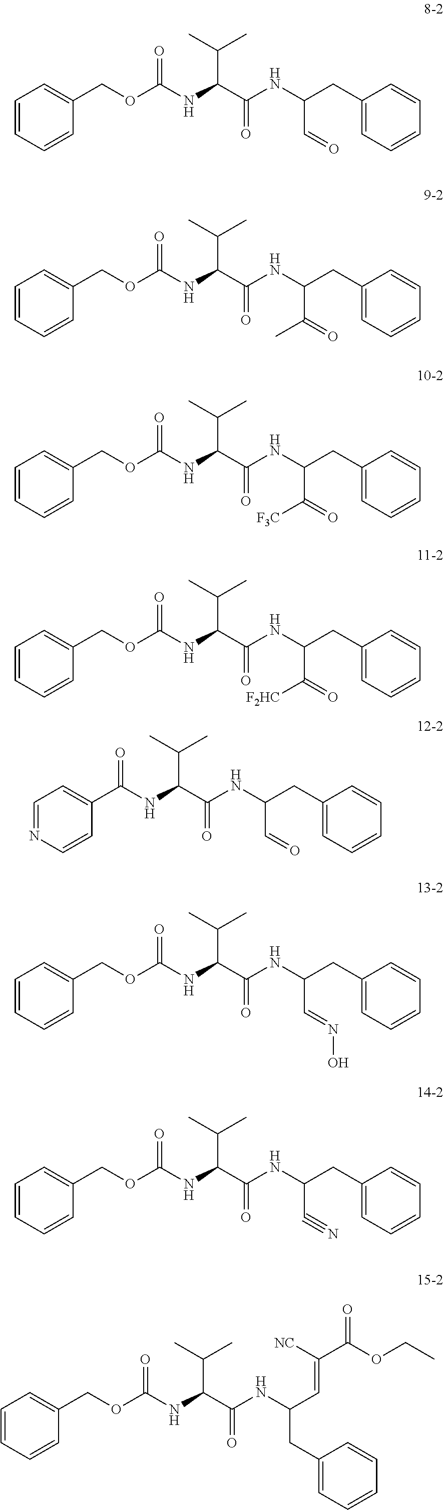

or a pharmaceutically acceptable salt thereof.

### Pharmaceutically Acceptable Salts

In some embodiments, a salt of a compound of Formula (I) of Formula (II), or any other compound described herein (e.g., an additional therapeutic agent), is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, 0-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds of Formula (I), or pharmaceutically acceptable salts thereof, are substantially isolated.

### Methods of Making Therapeutic Compounds

Compounds of Formula (I) or Formula (II), including salts thereof, can be prepared using known organic synthesis techniques and can be synthesized according to any of numerous possible synthetic routes. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Methods of Using Therapeutic Compounds

In some embodiments, the present disclosure provides a method of inhibiting enzymatic activity of a proteasome in a cell (e.g., at least one subunit of a proteasome in a cell), the method comprising contacting the cell with an effective amount of a compound of Formula (I) or Formula (II), or a pharmaceutically acceptable salt thereof. In some embodiments, the method comprises inhibiting one catalytic subunit within the proteasome. In some embodiments, the method comprises inhibiting two subunits within the proteasome.

In some embodiments, the present disclosure provides a method of inhibiting enzymatic activity of subunit β5 of a proteasome (PSMB5) in a cell, the method comprising contacting the cell with an effective amount of a compound of Formula (I) or Formula (II), or a pharmaceutically acceptable salt thereof

In some embodiments, the present disclosure provides a method of inhibiting enzymatic activity of subunit of a proteasome (PSMB9) in a cell, the method comprising contacting the cell with an effective amount of a compound of Formula (I) or Formula (II), or a pharmaceutically acceptable salt thereof.

In some embodiments, the present disclosure provides a method of inhibiting enzymatic activity of a subunit β5 and a subunit β1i of a proteasome in a cell, the method comprising contacting the cell with an effective amount of a compound of Formula (I) or Formula (II), or a pharmaceutically acceptable salt thereof.

In some embodiments, the proteasome is a 20S mammalian proteasome. In some embodiments, the cell is a cancer cell (e.g., multiple myeloma cell). In some embodiments, the cell is resistant to a proteasome inhibitor (e.g., bortezomib). In some embodiments, the cell is contacted in vitro, in vivo, or ex vivo. In some embodiments, the cell is contacted in vitro. In some embodiments, the cell is contacted in vivo. In some embodiments, the cell is contacted ex vivo.

In some embodiments, the present disclosure provides a method of inhibiting enzymatic activity of a proteasome in a subject (e.g., at least one subunit of a proteasome in a subject), the method comprising administering to the subject a therapeutically effective amount of a compound of Formula (I) or Formula (II), or a pharmaceutically acceptable salt thereof.

In some embodiments, the present disclosure provides a method of inhibiting enzymatic activity of subunit β5 of a proteasome (PSMB5) in a subject, the method comprising administering to the subject a therapeutically effective amount of a compound of Formula (I) or Formula (II), or a pharmaceutically acceptable salt thereof

In some embodiments, the present disclosure provides a method of inhibiting enzymatic activity of subunit β1i of a proteasome (PSMB9) in a subject, the method comprising administering to the subject a therapeutically effective amount of a compound of Formula (I) or Formula (II), or a pharmaceutically acceptable salt thereof.

In some embodiments, the present disclosure provides a method of inhibiting enzymatic activity of a subunit β5 and a subunit β1i of a proteasome in a subject, the method comprising administering to the subject a therapeutically effective amount of a compound of Formula (I) or Formula (II), or a pharmaceutically acceptable salt thereof.

In some embodiments, the subject is in need of administering the compound of Formula (I) or Formula (II), or a pharmaceutically acceptable salt thereof. In one example, the subject in need thereof is diagnosed with cancer by a treating physician. In this example, the compound of Formula (I) or Formula (II) inhibits proteasome (e.g., at least one subunit of the proteasome) is in the cancer cells of the subject.

In some embodiments, the present application provides a method of treating a cancer in a subject (e.g., a subject in need to cancer treatment), comprising administering to the subject (e.g., subject in need of cancer treatment) a therapeutically effective amount of a compound of Formula (I) or Formula (II), or a pharmaceutically acceptable salt thereof. In some embodiments, the subject in need of cancer treatment is a subject diagnosed with cancer by a treating physician.

In some embodiments, cancer is selected from the group selected from sarcoma, angiosarcoma, fibrosarcoma, rhabdomyosarcoma, liposarcoma, myxoma, rhabdomyoma, fibroma, lipoma, teratoma, lung cancer, breast cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney, adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, adrenal gland cancer, and neuroblastoma. Cancers may be solid tumors that may or may not be metastatic. Cancers may also occur, as in leukemia, as a diffuse tissue. In some embodiments, the cancer is resistant to a proteasome inhibitor (e.g., bortezomib-resistant cancer). In one example, the cancer is bortezomib-resistant multiple myeloma.

In some embodiments, the compound of Formula (I) or Formula (II), or a pharmaceutically acceptable salt thereof, may be administered to the subject in combination with at least one additional therapeutic agent.

In some embodiments, the therapeutic agent is an anticancer agent. Suitable examples of the anticancer agents include abarelix, ado-trastuzumab emtansine, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, emtansine, epirubicin, eribulin, erlotinib, estramustine, etoposide phosphate, etoposide, everolimus, exemestane, fentanyl citrate, filgrastim, floxuridine, fludarabine, fluorouracil, fruquintinib, fulvestrant, gefitinib, gemcitabine, gemtuzumab ozogamicin, goserelin acetate, histrelin acetate, ibritumomab tiuxetan, idarubicin, ifosfamide, imatinib mesylate, interferon α2a, irinotecan, ixabepilone, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, paclitaxel albumin-stabilized nanoparticle formulation, pamidronate, panitumumab, pegaspargase, pegfilgrastim, pemetrexed disodium, pentostatin, pertuzuma, pipobroman, plicamycin, procarbazine, quinacrine, rasburicase, rituximab, sorafenib, streptozocin, sulfatinib, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, volitinib, vorinostat, and zoledronate, or a pharmaceutically acceptable salt thereof. In some embodiments, the anticancer agent is a proteasome inhibitor (e.g., bortezomib, carfilzomib, or ixazomib).

In some embodiments, the additional therapeutic agent includes a pain relief agent (e.g., a nonsteroidal anti-inflammatory drug such as celecoxib or rofecoxib), an antinausea agent, a cardioprotective drug (e.g., dexrazoxane, ACE-inhibitors, diuretics, cardiac glycosides), a cholesterol lowering drug, a revascularization drug, a beta-blocker (e.g., acebutolol, atenolol, bisoprolol, metoprolol, nadolol, nebivolol, or propranolol), or an angiotensin receptor blocker (also called ARBs or angiotensin II inhibitors) (e.g., azilsartan, candesartan, eprosartan, irbesartan, losartan, olmesartan, telmisartan, or valsartan), or a pharmaceutically acceptable salt thereof.

In the method of treating cancer, the compound of Formula (I) or Formula (II) and the additional therapeutic agent may be administered to the subject simultaneously (e.g., in the same dosage form or in separate dosage forms), or consecutively (e.g., the additional therapeutic agent may be administered before or after the compound of Formula (I)).

In some embodiments, the compound of Formula (I) or Formula (II) may be administered to the subject in combination with one or more additional anti-cancer therapies selected from: surgery, biological therapy, radiation therapy, anti-angiogenesis therapy, immunotherapy, adoptive transfer of effector cells, gene therapy, and hormonal therapy.

### Pharmaceutical Compositions and Formulations

The present application also provides pharmaceutical compositions comprising an effective amount of a compound of Formula (I) or Formula (II), or a pharmaceutically acceptable salt thereof, and a pharmaceutically acceptable carrier. The pharmaceutical composition may also comprise any one of the additional therapeutic agents described herein, or a pharmaceutically acceptable salt thereof. In certain embodiments, the application also provides pharmaceutical compositions and dosage forms comprising any one the additional therapeutic agents described herein, or a pharmaceutically acceptable salt thereof, and a pharmaceutically acceptable carrier. The carrier(s) and excipient(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%400% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of Formula (I) or Formula (II) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound of Formula (I) or Formula (II) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent, such as any one of the anti-cancer agents described herein, in any one of amounts and dosage forms described herein.

### Definitions

At various places in the present specification, substituents of compounds of the present application are disclosed in groups or in ranges. It is specifically intended that various embodiments of the present application include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

As used herein, the term “tautomer” refers to compounds which are capable of existing in a state of equilibrium between two isomeric forms. Such compounds may differ in the bond connecting two atoms or groups and the position of these atoms or groups in the compound.

As used herein, the term “isomer” refers to structural, geometric and stereo isomers. As the compound of the present application may have one or more chiral centers, it is capable of existing in enantiomeric forms.

Throughout the definitions, the term “Cₙ₋ₘ” indicates a range which includes the endpoints, wherein n and m are integers and indicate the number of carbon atoms. Examples include C₁₋₄, C₁₋₆, and the like.

As used herein, the phrase “optionally substituted” means unsubstituted or substituted.

As used herein, the term “substituted” means that a hydrogen atom is removed and replaced by a substituent. It is to be understood that substitution at a given atom is limited by valency.

As used herein, the term “Cₙ₋ₘ alkyl”, employed alone or in combination with other terms, refers to a saturated hydrocarbon group that may be straight-chain (linear) or branched, having n to m carbons. Examples of alkyl moieties include, but are not limited to, chemical groups such as methyl, ethyl, n-propyl, isopropyl, n-butyl, tent-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ alkylene” means a bivalent saturated branched, or straight chain (linear) chemical group containing only carbon and hydrogen atoms, such as methylene, ethylene, n-propylene, iso-propylene, n-butylene, iso-butylene, sec-butylene, tert-butylene, n-pentylene, iso-pentylene, sec-pentylene and neo-pentylene. Alkylene groups can either be unsubstituted or substituted with one or more substituents. In some embodiments, alkylene groups include 1 to 9 carbon atoms (for example, 1 to 6 carbon atoms, 1 to 4 carbon atoms, or 1 to 2 carbon atoms).

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms. The term “Cₙ₋ₘ alkenylene” refers to a divalent alkenyl group.

As used herein, “Cₙ₋ₘ alkynyl” means a straight or branched chain chemical group containing only carbon and hydrogen , containing n to m carbon atoms and containing at least one carbon-carbon triple bond, such as ethynyl, 1-propynyl, 1-butynyl, 2-butynyl, and the like. In various embodiments, alkynyl groups can either be unsubstituted or substituted with one or more substituents. Typically, alkynyl groups will comprise 2 to 9 carbon atoms (for example, 2 to 6 carbon atoms, 2 to 4 carbon atoms, or 2 carbon atoms). The term “Cₙ₋ₘ alkynylene” refers to a divalent alkynyl group.

As used herein, the term “Cₙ₋ₘ alkoxy”, employed alone or in combination with other terms, refers to a group of formula —O—Cₙ₋ₘ alkyl, wherein the alkyl group contains n to m carbon atoms. Examplary alkoxy groups include, but are not limited to, methoxy, ethoxy, propoxy (e.g., n-propoxy and isopropoxy), butoxy (e.g., n-butoxy and tent-butoxy), and the like. In some embodiments, the alkoxy group has 1 to 6, 1 to 4, or 1 to 3 carbon atoms.

As used herein, “halo” refers to a halogen atom such as F, Cl, Br, or I. In some embodiments, a halo is F, Cl, or Br. In other embodiments, halo is F, Cl, or I. In other embodiments, halo is F, I, or Br.

As used herein, the term “Cₙ₋ₘhaloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘhaloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di Cₙ₋ₘ alkylamino” refers to a group of formula —N(alkyl)₂, wherein each alkyl group independently has n to m carbon atoms. In some embodiments, each alkyl group has 1 to 6, 1 to 4, or 1 to 3 carbon atoms. Examples of dialkylamino groups include, but are not limited to, N,N-methylehtylamino, N,N-diethylamino, N,N-propylethylamino, N,N-butylisopropylamino, and the like.

As used herein, “cycloalkyl” refers to non-aromatic saturated or unsaturated cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the non-aromatic cyclic hydrocarbon, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, or 12 ring-forming atoms. In some embodiments, the cycloalkyl is a 3-12 membered monocyclic or bicyclic cycloalkyl. In some embodiments, the cycloalkyl is a C₃₋₇ monocyclic cycloalkyl. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cyclooctyl, cyclooctenyl, and the like. In some embodiments, cycloalkyl is cyclopropyl, cyclobutyl, cyclopentyl, cyclohexyl, cyclooctyl, or cyclooctenyl. In some embodiments, the cycloalkyl is a cyclooctenyl ring fused with 1 or 2 benzene rings. In some embodiments, the cycloalkyl is a 3-8 membered or 3-7 membered monocyclic cycloalkyl group (e.g., C₃₋₈ or C₃₋₇ cycloalkyl).

In some embodiments, the cycloalkyl is a 8-12-membered bicyclic cycloalkyl. In some embodiments, the cycloalkyl is a 8-16-membered bicyclic or tricyclic cycloalkyl (e.g., C₈₋₁₆ cycloalkyl). In some embodiments, the cycloalkyl is unsaturated cyclic hydrocarbon group (i.e., the cycloalkyl contains at least one double bond).

As used herein, “heterocycloalkyl” or “aliphatic heterocycle” refers to non-aromatic saturated or unsaturated monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. In some embodiments, the heterocycloalkyl group is unsaturated (i.e., the heterocycloalkyl contains at least one double bond). Also included in the definition of heterocycloalkyl are moieties that have one or more aromatic rings fused (i.e., having a bond in common with) to the non-aromatic heterocycle, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. In some embodiments, the heterocycloalkyl is a 8-12-membered heterocycloalkyl (e.g., bicyclic heterocycloalkyl). In some embodiments, the heterocycloalkyl is a 8-16-membered heterocycloalkyl (e.g., bicyclic or tricyclic heterocycloalkyl). In some embodiments, the 8-12 membered bicyclic heterocycloalkyl is a 8-12 membered fused heterocycloalkylaryl group or a 8-12 membered fused heterocycloalkylheteroaryl group. In some embodiments, the heterocycloalkyl is a 9-12 membered bicyclic heterocycloalkyl. In some embodiments, the 9-10 membered bicyclic heterocycloalkyl is a 9-10 membered fused heterocycloalkylaryl group or a 9-10 membered fused heterocycloalkylheteroaryl group. The term “heterocycloalkylene” refers to a divalent heterocycloalkyl linking group.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 membered monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered heteroaryls are pyridyl, pyrazinyl, pyrimidinyl, triazinyl and pyridazinyl. The term “heteroarylene” refers to a divalent heteroaryl linking group.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

The term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl and the like. In some embodiments, aryl groups have from 6 to about 20 carbon atoms, from 6 to about 15 carbon atoms, or from 6 to about 10 carbon atoms. In some embodiments, the aryl group is phenyl. The term “arylene” refers to a divalent aryl linking group.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Example 1

### PSMB9 Mediates Resistance to Bortezomib in Multiple Myeloma

Introduction: Proteasomes are the chief degradation components of the ubiquitin proteasome system (UPS) and are comprised of several (a) structural and (B) catalytic subunits. Targeting the B5 subunit, with agents such bortezomib has proven highly successful in multiple myeloma patients. However, resistance to bortezomib invariably ensues with disease evolution resulting in relapse and mortality. The results presented herein highlight that in bortezomib resistant cells, proteasome function is upregulated with increased reliance on other β-catalytic subunits. The data shows that a novel association between the β5 subunit acting in concert with the βi proteasome subunit, to promote resistance to bortezomib. The data further shows that targeting β5 and β1i together would be lethal to bortezomib resistant multiple myeloma cells.

Methods: CD138+ cells from multiple myeloma patients, bortezomib-sensitive multiple myeloma cell lines (OPM2, KMS11 and U266) and their isogenic bortezomib resistant subclones were used in experiments. Fluorogenic peptide substrates were used to measure β-subunit-specific enzymatic activity. Gene disruption was conducted using 2 different shRNA hairpins for PSMB5 (β5) and PSMB9 (β9). Affymetrix HT12v4 gene expression array and NanoString mRNA quantification were used for gene expression profiling (GEP), followed by qPCR for confirmation. Novel dual β5-β1i proteasome inhibitors were synthesized and tested in vitro. Apoptosis was determined by annexin-V/proteasome inhibitor staining and MTS/CellTiter Glo assay used to determine cell viability.

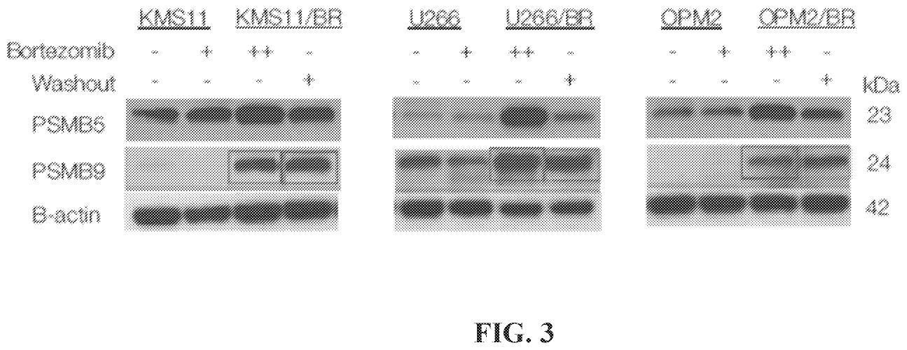

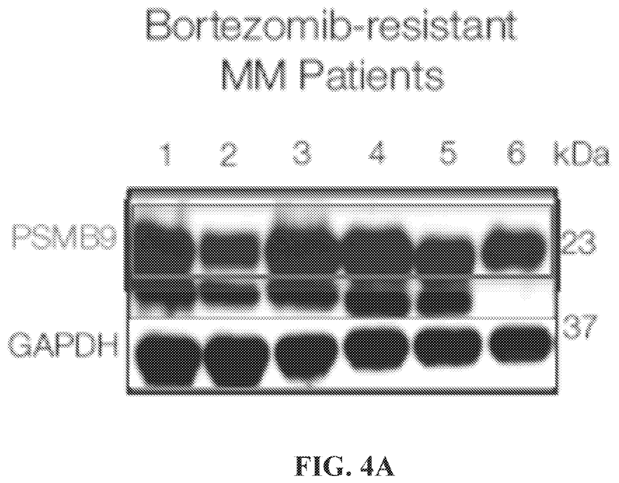

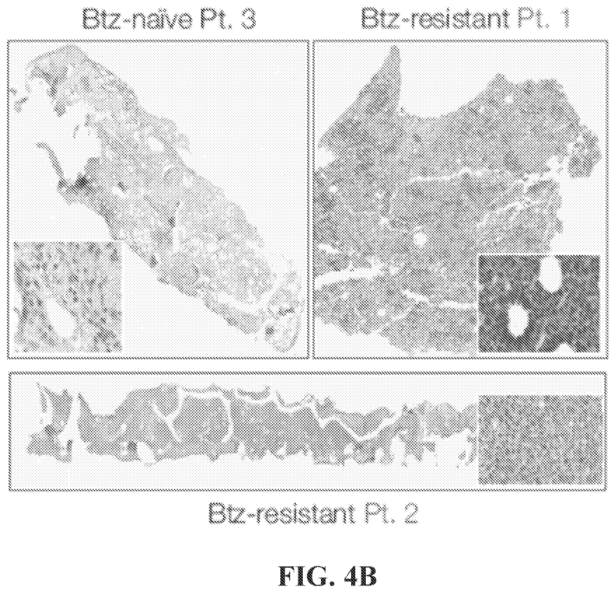

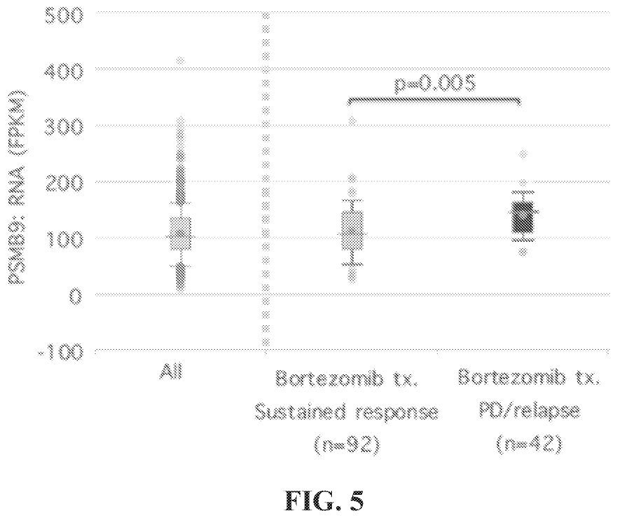

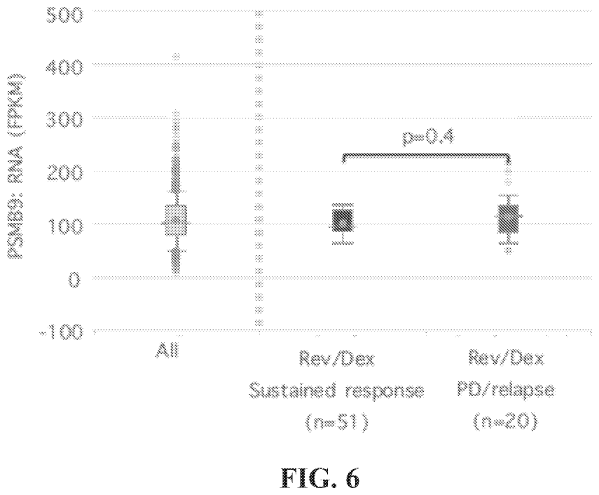

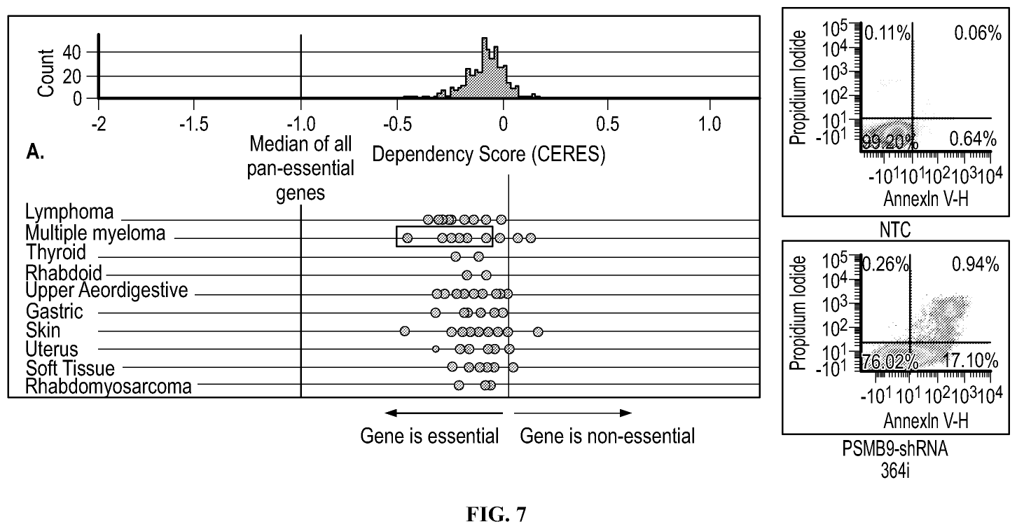

Results: FIG. 3 contains Western blot images showing that PSMB9 protein expression is upregulated in bortezomib-resistant multiple myeloma cells. Experimental data shown in FIGS. 4A and 4B shows that PSMB9 is markedly expressed in primary tumor cells from bortezomib-resistant myeloma patients. FIGS. 5 and 6 describe experimental results showing that PSMB9 mRNA is significantly lower in patients who respond to bortezomib-based therapy but does not correlate with response in patients on lenalidomide-based treatment. Furthermore, FIG. 7 shows that genetic interference of PSBM9 is lethal to multiple myeloma cells.

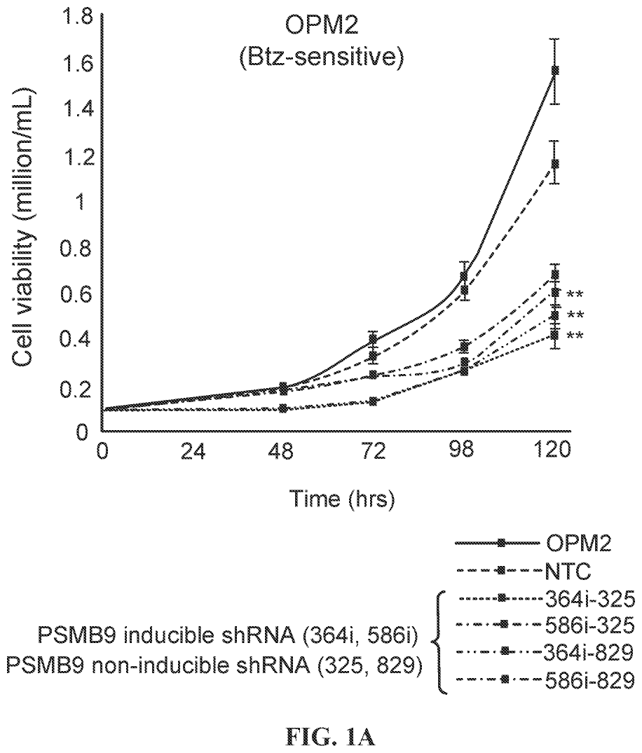

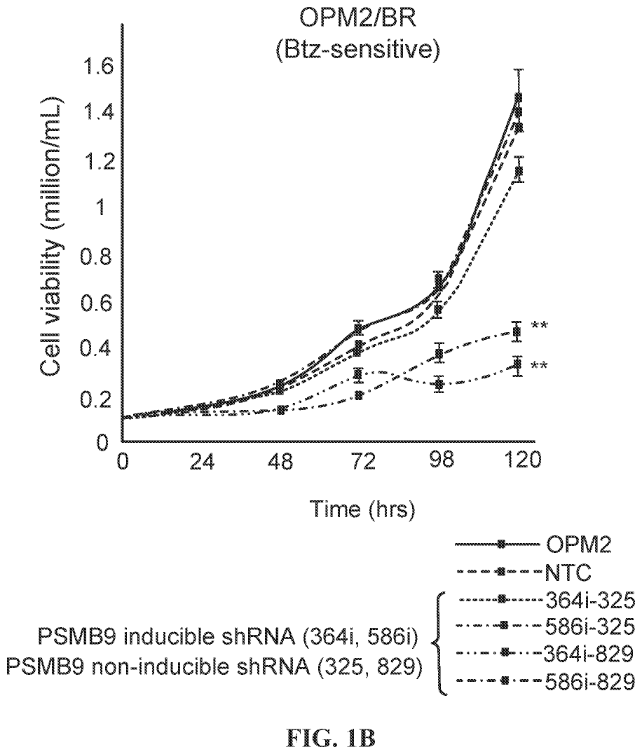

Bortezomib IC₅₀ was noted to be 284 nM in bortezomib resistant vs. 4 nM in bortezomib sensitive cells. Sanger seq. revealed no mutations in PSMB5 indicating that bortezomib should effectively bind the 135 subunit. B-subunit chymotrypic activity in bortezomib resistant vs. bortezomib sensitive cells was significantly increased (3.5 fold, p<0.001) but remained amenable to downregulation with bortezomib (as well as other proteasome inhibitors). This suggested that β5-subunit-independent mechanisms may account for increased proteasome function and resistance to bortezomib. GEP analysis revealed modulation of several genes; in particular, PSMB9 (β1i) in bortezomib resistant vs. bortezomib sensitive multiple myeloma cells. Proteomic analysis confirmed protein/enzymatic upregulation of PSMB5/β5 in conjunction with PSMB9/β1i in primary cells from bortezomib resistant multiple myeloma patients and bortezomib resistant multiple myeloma cell lines. Clinical significance of PSMB9 upregulation was queried using the MMRF CoMMPass database (IA10), with analysis in patients who received either bortezomib+dex (dexamethasone) or len (lenalidomide)+dex and did not respond to treatment. PSMB9 mRNA was significantly upregulated in bortezomib+dex non-responders (n=42) vs. responders (n=92), whereas in len+dex, this difference was not significant. We hypothesized that downregulation of PSMB9 would resensitize bortezomib resistant cells to bortezomib and noted a significant shift in IC₅₀ of in bortezomib resistant-PSMB9 shRNA transfected cells from 235 nM to 108 nM. While β1i knockdown alone was able to reduce bortezomib-resistant cell viability by about 25%, it was noted that concurrent disruption of β1i and 135 significantly reduce cell viability and proliferation. Targeted GEP in dual-B5/β1i knockdown bortezomib-resistant cells, showed more than 2-fold increase in CASP8, CASP9, CASP10, CDKN2A, CDKN1A,TNFRSF10B, FOXO4 and RNF43 (apoptosis genes) and decrease in CCB1, CCNA2, WNT5A, SKP2, CDC6 and FEN1 (cell cycle genes) vs. scramble-transfected controls. Indeed, both bortezomib-sensitive and bortezomib-resistant multiple myeloma cells demonstrated significantly compromised growth capacity and viability (dual knockdown 135/β1i bortezomib-resistant cells: 0.32 million/mL vs. Scr bortezomib-resistant cells: 1.6 million/mL) (See FIGS. 1A and 1B).

The dual-β5 and β1i chemical inhibitors of the present disclosure display binding affinity for both β5 and β1i. In vitro screening of these compounds showed more than 2 fold lower EC₅₀ in bortezomib-resistant vs. bortezomib-sensitive multiple myeloma cell lines.

Conclusions: The data of Example 1 shows that upregulation of PSMB9/β1i and PSMB5/β5 is associated with resistance to bortezomib. These findings were confirmed in both multiple myeloma cell lines as well as in primary multiple myeloma cells from bortezomib-resistant patients. Moreover, increased PSMB9 mRNA expression in patients non-responding bortezomib+dex but not len+dex multiple myeloma patients highlights this as a resistance mechanism unique to bortezomib. Using direct genetic disruption or dual-chemical inhibitors directed toward both β1i and β5 induced lethality in bortezomib-resistant (as well as bortezomib-sensitive) multiple myeloma cells.

### Example 2

### Dual-targeting of β5 and β1i-Proteasome Catalytic Subunits is Lethal to Waldenström Macroglobulinemia Cells.

Introduction: Waldenstrom Macroglobulinemia (WM) cells secrete high amounts of IgM and rely on the proteasome for optimal protein homeostasis. Clinically, disruption of the protein homeostasis using proteasome inhibitors (PI) such as bortezomib results in induction of remission in over 85% of patients. The catalytic activity of the proteasome rely on its multiple subunit and the current generation of PI are specifically designed to target its β5 subunit. Despite initial responses all patients with WM develop resistance to β5-targeted therapeutics. Preclinical models of PI resistance identified upregulation of the β1i subunit catalytic activity. As shown in Example 1, concurrent targeting of both β1i and β5 subunits could overcome resistance or enhance anti-WM activity. The chemical inhibitor co-targeting β5 and β1i-proteasome catalytic subunits and its effect in WM cells are described in this example.

Methodology: BWCM.1, MWCL-1 and RPCI-WM1 and isogenic PI-resistant subclones were used in experiments. WM cell viability determined by 72 hour CellTiter Glo assay. Proteasome enzyme activity was estimated by fluorogenic substrates specific for β5 and β1. Protein expression was determined by western blotting. Apoptosis was assessed by annexin-V/PI staining.

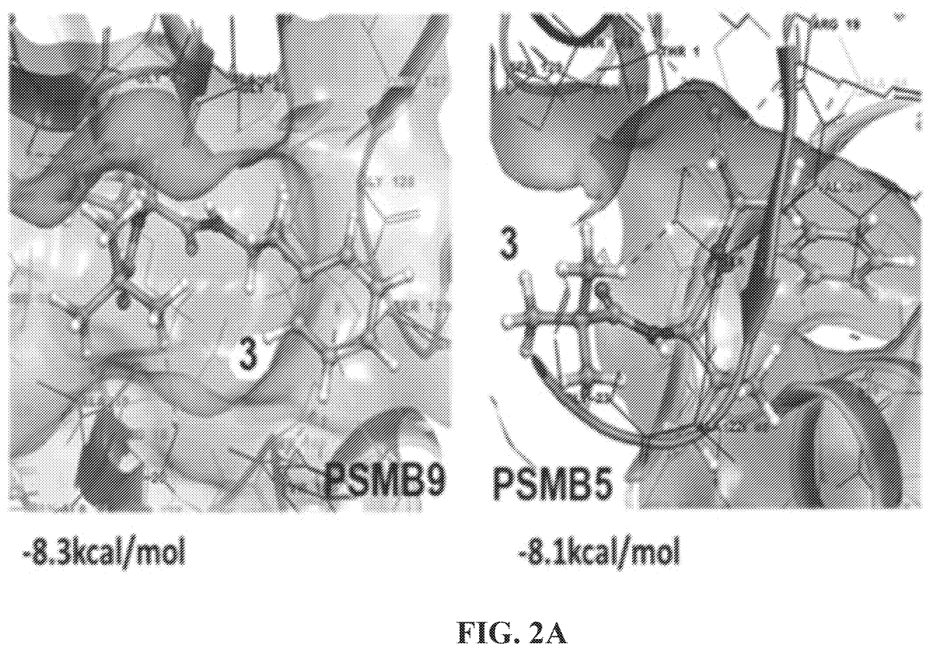

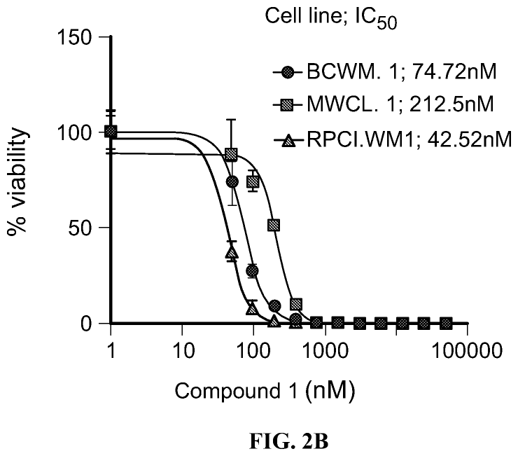

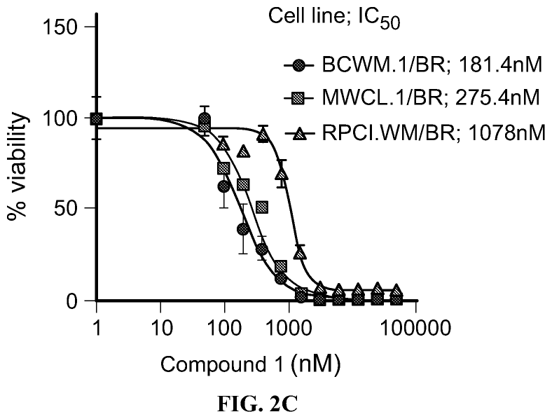

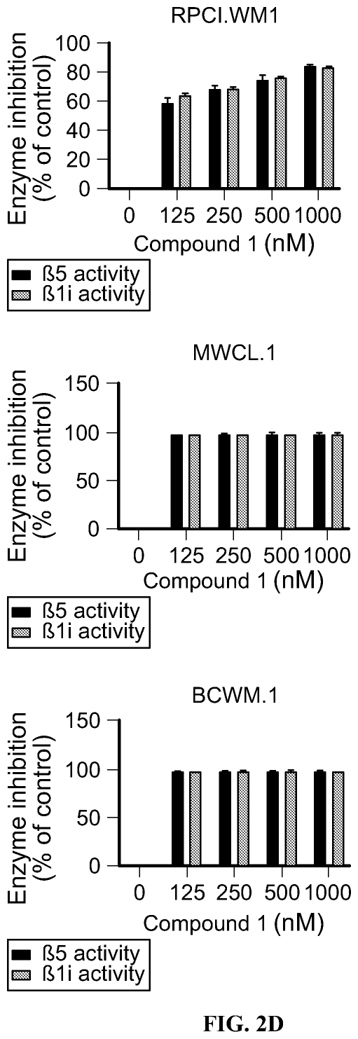

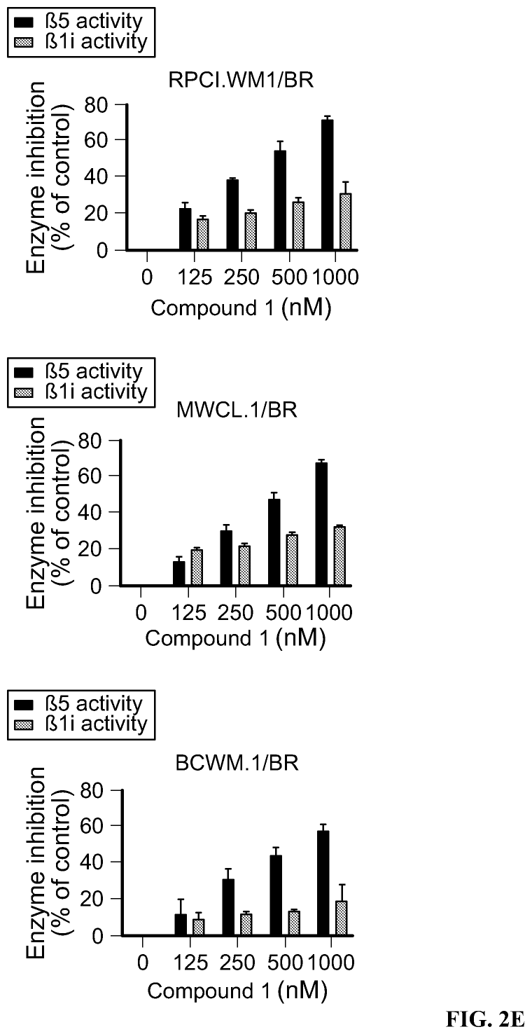

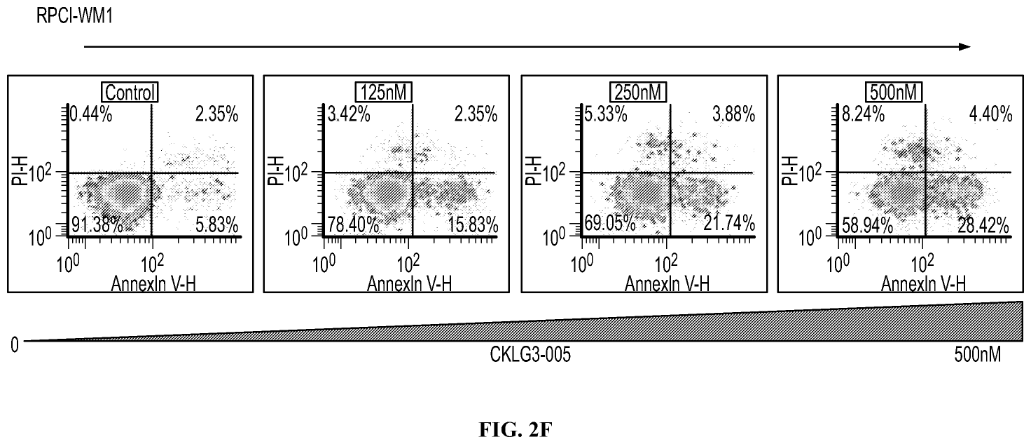

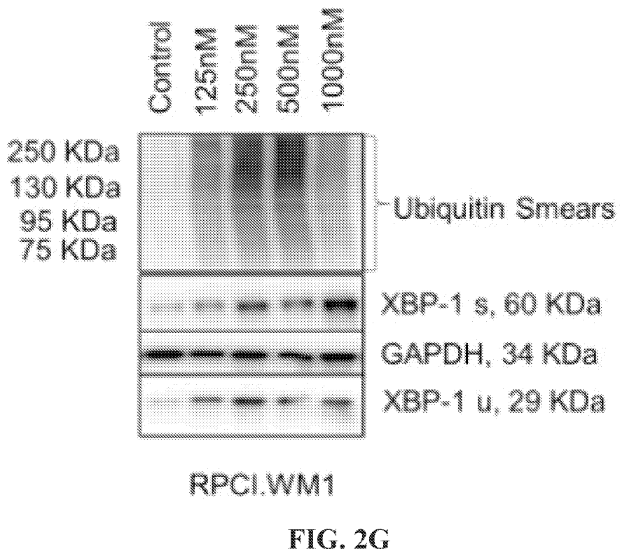

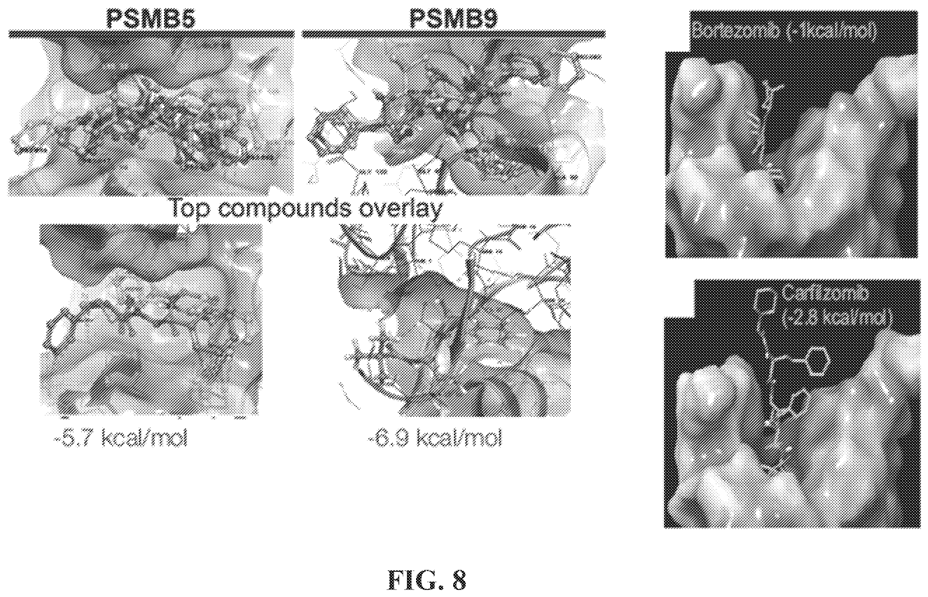

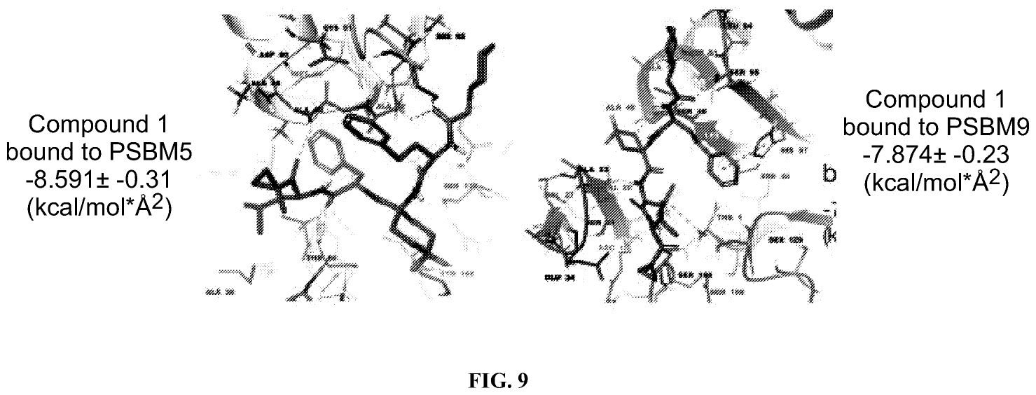

Results: Chemical compounds capable of inhibiting enzymatic activity of both β5 and β1 were prepared. Compound 1 (FIG. 2A)) showed remarkable cytotoxic activity in WM cell lines; with a median EC₅₀ of 196.95 nM. (FIGS. 2B, 2C). In silico docking for Compound 1 to PSMB5 and PSMB9 showed a binding energy of −8.1 Kcal/mol and −8.3Kcal/mol, respectively (FIGS. 8 and 9 contain images showing binding affinity of dual-β5/β1i small molecule inhibitors (or current PI) to PSMB5 and PSMB9, and images of Compound 1 bound to PSBM5 and PSBM9). Comparatively, bortezomib binds PSMB5 and PSMB9 at −7.8 and −1 Kcal/mol, respectively; thus, showing superior binding affinity of Compound 1 over bortezomib. In vitro proteasome enzyme activity at the β5 and β1 subunits was examined in all WM cell lines +/−Compound 1 (with bortezomib serving as a comparator). Indeed, we noted that in wildtype WM cells, median % inhibition of β5 and β1 enzymatic activity was 97.1% and 97.4%, respectively. In bortezomib-resistant isogenic WM clones the median % inhibition of β5 and β1 enzymatic activity was 30.4% and 18.8%, respectively (Compound 1; 250 nM) (FIGS. 2D, 2E). As anticipated, bortezomib however did not decrease β1 activity but only β5 enzymatic function. It was also noted that Compound 1 induced robust apoptosis in WM cells, however at concentrations similar to those required to inhibit β5 and β1 activity, apoptosis was most prominent in RPCI-WM1 cells (developed from a bortezomib-resistant WM patient) (FIG. 2F). Apoptotic cell death was also accompanied by induction of the ER-stress response as a dose-dependent increase in XBP-1 and XBP-1s proteins and polyubiquitinated protein was evident (FIG. 2G).

### Example 3

### Bioactivity of Dual β5 and β1i-Proteasome Inhibitors.

Results of IC₅₀ measurements in the MTS assay for compound 3 are shown in Table 1.

TABLE 1

| Compound | BCWM.1 | BCWM.1/BR | MWCL.1 | MWCL.1/BR | RPCI.WM1 | RPCI.WM1/BR |
|---|---|---|---|---|---|---|
| Bortezomib | 8.6 | 17.6 | 10.2 | 16.7 | 3.0 | 114.2 |
| (control), nM | | | | | | |
| Cmpd 3, nM | 466.7 | 885 | 999.2 | 1191 | 190.3 | 2494 |

### Example 4

### Bioactivity of Dual β5 and β1i-Proteasome Inhibitors

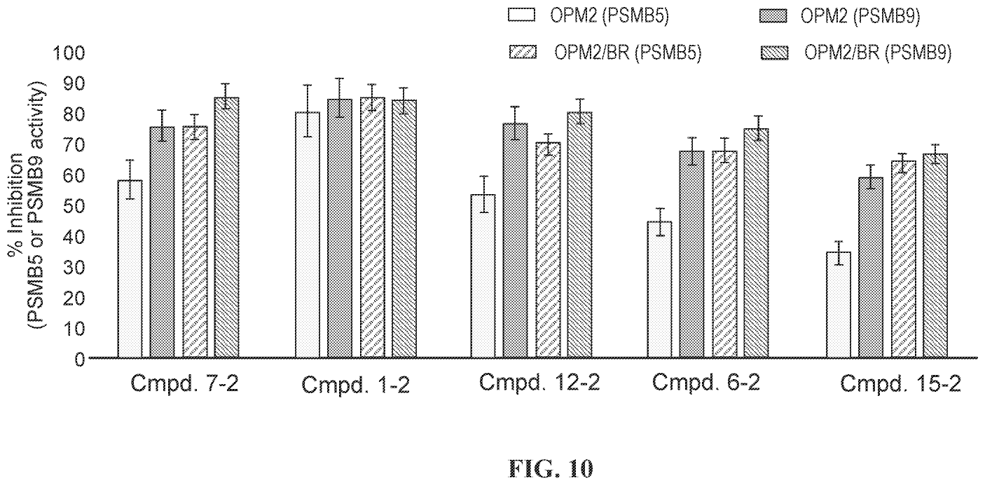

FIG. 10 shows in vitro enzymatic and anti-tumor potency of dual-β5/β1i small molecule inhibitors. The results were shown for multiple myeloma cell lines (OPM2, OPM2/BR). Results of IC₅₀ measurement for tested compounds are shown in Table 2.

TABLE 2

| | | |
|---|---|---|
| Compound | B1i ≥ IC₅₀ | B1i ≥ IC₅₀ |

| | | | |
|---|---|---|---|
| 12-2 | 5 | μM | 5 μM |
| 1-2 | 1 | μM | 5 μM |
| 15-2 | >10 | μM | 5 μM |
| 7-2 | 5 | μM | 5 μM |
| 6-2 | 1 | μM | 5 μM |

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1-61. (canceled)

62. A compound of Formula (I): 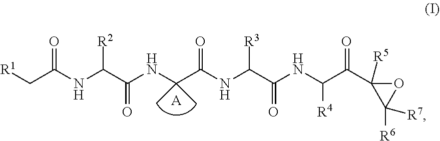
or a pharmaceutically acceptable salt thereof, wherein:
R¹ is 4-10 membered heterocycloalkyl, optionally substituted with 1, 2, or 3 substituents independently selected from R^{a};
R², R³, and R⁴ are each independently selected from H, hydroxymethyl, (imidazol-4-yl)methyl, 3-guanidinopropyl, 4-aminobutyl, 3-aminopropyl, carboxymethyl, 2-carboxyethyl, 1-hydoxyethyl, 2-hydroxyethyl, carbamylmethyl, 2-carbamyl ethyl, thiomethyl, 2-thioethyl, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;
ring A is selected from 4-7 membered heterocycloalkyl ring and C₃₋₇ cycloalkyl ring, each of which is optionally substituted by 1, 2, or 3 substituents independently selected from R^{a};
R⁵, R⁶, and R⁷ are each independently selected from H and C₁₋₃ alkyl, wherein said C₁₋₃ alkyl is optionally substituted with 1, 2, or 3 substituents independently selected from R^{a}; and
each R^{a} is independently selected from C₁₋₃ alkyl, C₁₋₃ haloalkyl, OH, NO₂, CN, halo, C₁₋₃ alkoxy, C₁₋₃ haloalkoxy, NH₂, C₁₋₃ alkylamino and di(C₁₋₃ alkyl)amino.

63. The compound of claim 62, wherein the compound of Formula (I) has formula: 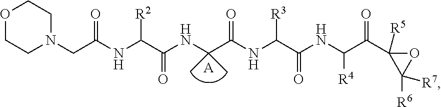
or a pharmaceutically acceptable salt thereof.

64. The compound of claim 62, wherein R², R³, and R⁴ are each independently selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl.

65. The compound of claim 62, wherein:
R⁴ is isobutyl; and
R² and R³ are each independently selected from H, phenylmethyl, 2-phenylethyl.

66. The compound of claim 65, wherein:
R² is 2-phenylethyl; and
R³ is phenylmethyl.

67. The compound of claim 65, wherein:
R² is 2-phenylethyl; and
R³ is H.

68. The compound of claim 62, wherein R⁵, R⁶, and R⁷ are each independently selected from H and C₁₋₃ alkyl.

69. The compound of claim 68, wherein
R⁵ is C₁₋₃ alkyl; and
R⁶ and R⁷ are each H.

70. The compound of claim 69, wherein the compound of Formula (I) has formula: 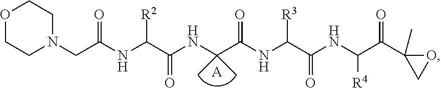
or a pharmaceutically acceptable salt thereof.

71. The compound of claim 62, wherein ring A is 4-7 membered heterocycloalkyl ring selected from aziridinyl, oxetanyl, pyrrolidinyl, tetrahydrofuranyl, tetrahydrothiofuranyl, morpholinyl, thiomorpholinyl, piperidinyl, piperazinyl, tetrahydropyranyl, 2-azaspiro[3.3]heptyl, and 2-oxaspiro[3.3]heptyl, optionally substituted by 1, 2, or 3 substituents independently selected from R^{a}.

72. The compound of claim 62, wherein ring A is C₃₋₇ cycloalkyl ring selected from cyclopropyl, cyclobutyl, cyclopentyl, cyclohexyl, cycloheptyl, and spiro[3.3]heptyl, each of which is optionally substituted by 1, 2, or 3 substituents independently selected from R^{a}.

73. The compound of claim 62, wherein the compound of Formula (I) has formula: 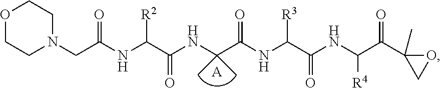
or a pharmaceutically acceptable salt thereof, wherein:
R², R³, and R⁴ are each independently selected from H, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;
ring A is C₃₋₇ cycloalkyl ring, optionally substituted by 1, 2, or 3 substituents independently selected from R^{a}; and
each R^{a} is independently selected from halo, C₁₋₃ alkyl, C₁₋₃ haloalkyl, C₁₋₃ alkoxy, and C₁₋₃ haloalkoxy.

74. The compound of claim 73, wherein
R⁴ is isobutyl;
R² and R³ are each independently selected from H, phenylmethyl, and 2-phenylethyl;
ring A is selected from cyclohexyl and spiro[3.3]heptyl, each of which is optionally substituted by 1, 2, or 3 halo.

75. The compound of claim 62, wherein the compound of Formula (I) is selected from any one of the following compounds: 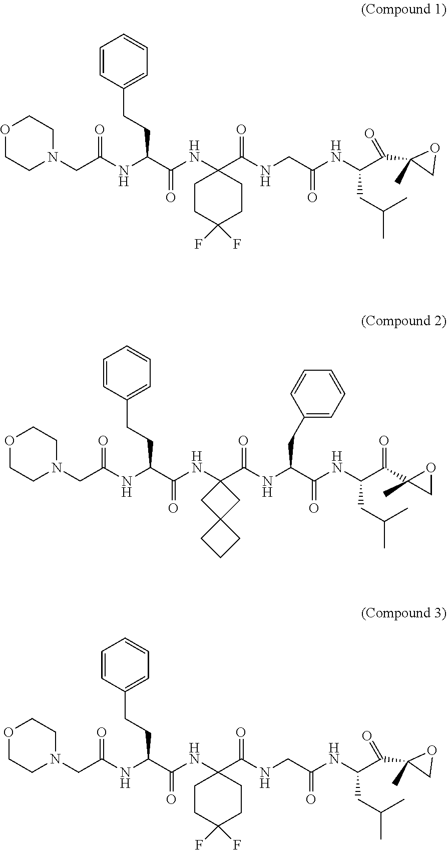
or a pharmaceutically acceptable salt thereof.

76. A pharmaceutical composition comprising a compound of claim 62, or a pharmaceutically acceptable salt thereof, and a pharmaceutically acceptable carrier.

77. A method of inhibiting enzymatic activity of a subunit β5 and a subunit β1i of a proteasome in a cell, the method comprising contacting the cell with an effective amount of a compound of claim 62, or a pharmaceutically acceptable salt thereof.

78. A method of treating multiple myeloma in a subject, the method comprising administering to the subject a therapeutically effective amount of a compound of claim 62, or a pharmaceutically acceptable salt thereof.

79. A compound of Formula (II): 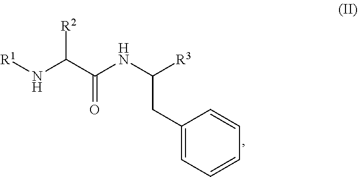
or a pharmaceutically acceptable salt thereof, wherein:
R¹ is selected from C(O)R^{a1}, C(O)OR^{a1}, and S(O)₂R^{a1};
R² is selected from H, hydroxymethyl, (imidazol-4-yl)methyl, 3-guanidinopropyl, 4-aminobutyl, 3-aminopropyl, carboxymethyl, 2-carboxyethyl, 1-hydoxyethyl, 2-hydroxyethyl, carbamylmethyl, 2-carbamylethyl, thiomethyl, 2-thioethyl, methyl, ethyl, n-propyl, isopropyl, sec-butyl, isobutyl, n-butyl, n-hexyl, 2-(methylthio)ethyl, (methylamino)methyl, phenylmethyl, 2-phenylethyl, (4-hydroxyphenyl)methyl, and (indol-3-yl)methyl;
R³ is selected from C(O)R^{b1}, CN, C(═NR^{e1})R^{b1}, and a group of formula: 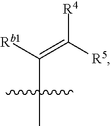
R⁴ and R⁵ are each independently selected from C(O)OR^{c1}, S(O)₂R^{c1}, C(O)R^{c1}, C(O)NR^{c1}R^{d1}, S(O)₂NR^{c1}R^{d1}, CN, and NO₂;
each R^{a1} is selected from C₁₋₆ alkyl, C₁₋₆ haloalkyl, C₂₋₆ alkenyl, C₂₋₆ alkynyl, C₆₋₁₀ aryl, 5-14 membered heteroaryl, C₆₋₁₀ aryl-C₁₋₄ alkylene, and (5-10 membered heteroaryl)-C₁₋₄ alkylene, each of which is optionally substituted with 1, 2, or 3 substituents independently selected from R^{g};
each R^{b1} is selected from H, C₁₋₃ alkyl, and C₁₋₃ haloalkyl;
R^{e1} is selected from H, C₁₋₃ alkyl, C₁₋₃ alkoxy, OH, and CN;
each R^{c1} and R^{d1} are independently selected from H, C₁₋₃ alkyl, and C₁₋₃ haloalkyl; and
each R^{g} is independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino.

80. A method of inhibiting enzymatic activity of a subunit β5 and a subunit β1i of a proteasome in a cell, the method comprising contacting the cell with an effective amount of a compound of claim 79, or a pharmaceutically acceptable salt thereof.

81. A method of treating multiple myeloma in a subject, the method comprising administering to the subject a therapeutically effective amount of a compound of claim 79, or a pharmaceutically acceptable salt thereof.
